(19) United States
(12) Patent Application Publication
(10) Pub. No.: US 2005/0220828 A1
(43) Pub. Date: Oct. 6, 2005

(54) Treatment of keratinous tissue

(75) Inventors: Michael Jason Ullom, Mason, OH (US); Paul Robert Tanner, Lebanon, OH (US); Reuben Earl Oder III, Lebanon, OH (US); Erica Heidi Hollman, Kenosha, WI (US); Nancy Coultrip Dawes, Cincinnati, OH (US); Debra Gay Gehring, Cincinnati, OH (US); Karl Anton Bakker, Chicago, IL (US); Ty Eric Martin, Loveland, OH (US); Peter James Hargraves, Cincinnati, OH (US); Tanya Nicole Blasko, Cincinnati, OH (US)

Correspondence Address: THE PROCTER & GAMBLE COMPANY;INTELLECTUAL PROPERTY DIVISION, WINTON HILL TECHNICAL CENTER - BOX 161, 6110 CENTER HILL AVENUE, CINCINNATI, OH, 45224, US

(73) Assignee: The Procter & Gamble Company

(21) Appl. No.: 10/798,473

(22) Filed: Mar. 11, 2004

Related U.S. Application Data
(60) Provisional application No. 60/516,502, filed on Oct. 31, 2003.
(60) Provisional application No. 60/516,523, filed on Oct. 31, 2003.

(51) Int. Cl. A61K 7/00

(52) U.S. Cl. 424/401

(57) ABSTRACT

A method of treating keratinous tissue is disclosed. The treatment utilizes at least one topical composition for application to keratinous tissue, a sachet containing the topical composition and a receptacle with heating system for releasably receiving at least one sachet.

### FIELD OF THE INVENTION

This invention relates to a method of treating keratinous tissue. The treatment utilizes at least one topical composition for application to keratinous tissue, a sachet containing the topical composition and a receptacle with heating system for releasably receiving at least one sachet.

### BACKGROUND OF THE INVENTION

Many topically applied products currently available to consumers are directed primarily to improving the health and/or physical appearance of the skin. Many of these skin care products are directed to delaying, minimizing or even eliminating skin wrinkling, skin discolorations, and other histological changes typically associated with the aging and, or environmental damage to human skin. Other topically applied products are pharmaceuticals, designed to treat specific diseases via the keratinous tissue.

Consumers prefer topically applied products since they are not only effective, but also safe and pleasant to use. The experience of topical application is enhanced when the topically applied product is warmed or heated. For example, when a warmed moisturizing lotion is applied to the skin of the face or body, the sensation is perceived by users as more pleasing than application of a cold lotion. The experience of applying the lotion, then, becomes a motivating factor in using the lotion and the lotion is applied more frequently than would be the case if it was applied cold. Regular use of topical products ensures that the maximum benefit of the actives is achieved.

Many topical compositions are reduced in viscosity when warmed. The reduced viscosity can result in a composition that flows more readily onto the skin, hair or nails. Petroleum jelly is an example of such a product. When warmed, the petroleum jelly becomes less viscous and the user can apply a thinner coat with less force. When the petroleum jelly cools, it returns to its original rheology, and a thin, but complete, coat remains on the surface to which it has been applied.

It is, of course, possible for topical compositions to become so thin that they are not aesthetically pleasing, are more difficult to apply and, when spilled, quickly soil the surrounding area. For these reasons, it is important that the temperature to which the topical compositions are heated is within a controlled range. This controlled range varies with the specific composition being heated.

It is known to warm or heat topically applied products via delivery of fluid product from reservoirs. However, many topically applied products are easily degraded by heat, especially by repeated heating. In addition, the time required for heating all of the product in a large reservoir is significant. Lotion users today are often limited with regard to the time allotted to personal care and a warming step may be skipped when the user is in a hurry.

Warmer-holders for filled packets of fluid are known in the art. Such warmer-holders include a heater assembly, to which power is provided by electrical plug-pins that plug into power socket outlets.

Based on the foregoing, there is a continuing need to provide topical compositions in a form which will provide a pleasant warmth while being quick to prepare and easy to use.

### SUMMARY OF THE INVENTION

This application discloses a method for treating keratinous tissue. The method comprises as a first step, warming a topical composition contained in a sachet using a receptacle. The receptacle comprises: walls defining a cavity for releasably receiving at least one sachet; a heating system associated with one or more of the walls of the cavity; and a power source operatively associated with the heating system. The power source supplies sufficient energy to the heating system to warm the composition inside the sachet to a target temperature sufficient to provide easy application to the keratinous tissue but not so fluid to run off the surface when applied. Generally the temperature is from about 30° C. to 65° C. The warmed composition is removed from the sachet and applied to keratinous tissue.

In addition, the application discloses a receptacle for warming a topical composition contained in a sachet. The receptacle comprises walls defining a cavity for releasably receiving at least one sachet; and a heating system associated with one or more of the cavity walls. The heating system achieves and maintains a target temperature of the topical composition. In one embodiment the heating system is activated by insertion of the sachet. In another embodiment the receptacle is controlled by a monitor such that when a target temperature for the topical composition, appropriate to the application of the topical composition, is achieved, the heating system maintains that target temperature for a predetermined period. Lastly, the receptacle comprises a power source operatively associated with the heating system for supplying energy to the heating system.

Also disclosed is a kit for treatment of keratinous tissue. The kit comprises at least one topical composition, at least one sachet comprising surfaces defining one or more chambers for containing the composition and a receptacle for releasably receiving at least one sachet. The receptacle comprises walls defining a cavity for releasably receiving the sachet(s), a heating system associated with one or more of the walls; and a power source operatively associated with the heating system. The composition is warmed when the sachet is inserted into the cavity of the receptacle.

Also disclosed herein is a skin care regimen that comprises selecting a sachet from a sachet assortment, inserting the sachet into a receptacle, as described above, for releasably receiving the sachet, and activating the heating system operatively associated with the receptacle, warming the composition contained in the sachet. After warming, the sachet is opened and the warmed composition is applied to a user's skin. This regimen is optionally, but preferably, followed at least once per day.

### BRIEF DESCRIPTION OF THE DRAWINGS

FIG. 1a is a perspective view of an embodiment of a receptacle with a sachet inserted.

FIG. 1b is a right side view of the embodiment of FIG. 1a, with no sachet inserted.

FIG. 1c is a top plan view of the embodiment of FIG. 1a.

FIG. 1d is an exploded view of the embodiment of FIG. 1a, with no sachet inserted.

FIG. 2a is a front view of a second embodiment of a battery-powered receptacle.

FIG. 2b is a right side view of the embodiment of FIG. 2a.

FIG. 2c is a cross sectional view of the embodiment of FIG. 2a taken along line A-A.

FIG. 2d is a top plan view of the embodiment of FIG. 2a.

FIG. 2e is a back plan view of the embodiment of FIG. 2a.

FIG. 3a is a perspective view of a third embodiment of a receptacle designed to receive a plurality of sachets.

FIG. 3b is a top plan view of the embodiment shown in FIG. 3a.

FIG. 4a is a perspective view of a fourth embodiment of a receptacle inserted into a docking station.

FIG. 4b is a top plan view of the receptacle with docking station shown in FIG. 4a.

FIG. 4c is a cross sectional view of the receptacle with docking station shown in FIG. 4a taken along line A-A of FIG. 4b.

FIG. 4d is a left side plan view of the receptacle with docking station shown in FIG. 4a.

FIG. 4e is a front plan view of the receptacle with docking station shown in FIG. 4a.

FIG. 5a is a front plan view of a first embodiment of a sachet.

FIG. 5b is a perspective view of a sachet as shown in FIG. 5a.

FIG. 5c is a bottom plan view of a sachet as shown in FIG. 5a.

FIG. 5d is a right side view of a sachet as shown in FIG. 5a.

FIG. 5e is a cross sectional view of the sachet shown in FIG. 5a, taken along line A-A of FIG. 5b.

FIG. 6a is a front plan view of a second embodiment of a sachet having two chambers.

FIG. 6b is a cross sectional view of a sachet as shown in FIG. 6a taken along line A-A and showing the contained compositions.

FIG. 7a is a front plan view of a third embodiment of the sachet also having two chambers.

FIG. 7b is a left side view of the embodiment of FIG. 7a.

FIG. 7c is a perspective view of the embodiment shown in FIG. 7a.

FIG. 8a is a front plan view of a fourth embodiment of a sachet having a contact point to activate the heater system.

FIG. 8b is a side view of the sachet of FIG. 8a.

FIG. 8c is perspective view of the sachet of FIG. 8a.

FIG. 9a is a perspective view of a fourth embodiment of a receptacle having two heating elements and two heat sinks.

FIG. 9b is a front plan view of the embodiment of FIG. 9a.

FIG. 9c is a cross sectional view of the embodiment of FIG. 9a, taken along line B-B of FIG. 9b.

### DETAILED DESCRIPTION OF THE INVENTION

All percentages and ratios used herein are by weight of the total composition and all measurements made are at 25° C., unless otherwise designated.

The compositions of the present invention can comprise, consist essentially of, or consist of, the essential components as well as optional ingredients described herein. As used herein, “consisting essentially of” means that the composition or component may include additional ingredients, but only if the additional ingredients do not materially alter the basic and novel characteristics of the claimed compositions or methods.

The term “keratinous tissue,” as used herein, refers to keratin-containing layers disposed as the outermost protective covering of mammals which includes, but is not limited to, skin, hair, toenails, fingernails, cuticles, hooves, lips, etc.

The term “topical application”, as used herein, means to apply or spread the compositions of the present invention onto the surface of the keratinous tissue.

The term “dermatologically acceptable,” as used herein, means that the compositions or components described are suitable for use in contact with mammalian keratinous tissue without undue toxicity, incompatibility, instability, allergic response, and the like.

The term “flexible” as used herein, means “easily collapsed, flattened, deformed or bent by the hand or fingers”.

The term “rigid” as used herein, means “not easily collapsed, flattened, deformed or bent by the hand or fingers”.

The term “heating system”, as used herein refers to all components used either to transfer energy from the energy source to the contents of the sachet or to indicate when the target temperature has been achieved. Examples are: the heating element associated with the cavity wall adjacent to the power source and various other components.

The term “monitor”, as used herein means a device that controls all functions of the heating system.

The term “microprocessor”, as used herein, means a computer whose entire central processing unit (CPU) is contained on an integrated chip, or a plurality of integrated chips.

The term “microcontroller”, as used herein, means a microprocessor on a single integrated circuit intended to operate as an embedded system. In addition to a CPU, a microcontroller typically includes small amounts of RAM and PROM, timers, and input/out.

The term “heat sink” as used herein refers to a piece of thermally conductive material designed to absorb, distribute, or transfer heat from a heat source.

The term “sachet”, as used herein, refers to a packet or pouch having fluid impervious walls that can be filled with a composition containing a topical composition or ingredient.

The term “unit dose” as used herein means an amount of the composition sufficient for a single application or treatment.

The term “safe and effective amount” as used herein means an amount of a compound or composition sufficient to significantly induce a positive benefit, preferably a positive keratinous tissue appearance or feel benefit, including independently or in combination the benefits disclosed herein, but low enough to avoid serious side effects (i.e., to provide a reasonable benefit to risk ratio, within the scope of sound judgment of the skilled artisan).

The term “timer” as used herein means a timepiece that measures a time interval and signals its end. It can also act as the regulator that starts/activates or stops/deactivates a mechanism or process at a set time.

The term “protrusion” as used herein means a projection, i.e., a part that juts out from the body of an object such as a sachet.

The term “contact point” as used herein refers to any portion of the sachet that activates the heating system by communication with a switch.

The term “operatively associated” as used herein means to join or connect together to produce an appropriate effect.

Topical Composition

The compositions for use in the present invention are topically applied. The topical compositions disclosed are useful for regulating keratinous tissue. Regulation of keratinous tissue, especially human skin, is often required due to conditions that may be induced or caused by factors internal and/or external to the body. For instance, “regulating skin” includes prophylactically regulating and/or therapeutically regulating skin condition, and may involve one or more of the following benefits: thickening (i.e., building the epidermis and/or dermis layers of the skin and/or the subcutaneous layers such as fat and muscle and where applicable the keratinous layers of the nail and hair shaft) to reduce atrophy (e.g., of the skin); increasing the convolution of the dermal-epidermal border; and reducing non-melanin skin discoloration such as under eye circles, blotching (e.g., uneven red coloration due to, e.g., rosacea) (hereinafter referred to as “red blotchiness”), sallowness (pale or yellow color), discoloration caused by telangiectasia or spider vessels, discolorations due to melanin (e.g., pigment spots, age spots, uneven pigmentation, hyperpigmentation, such as post-inflammatory hyperpigmentation) and other chromophores in the skin (e.g., lipofuscin, protein crosslinks such as those that occur with glycation, and the like). As used herein, prophylactically regulating skin condition includes delaying, minimizing and/or preventing visible and/or tactile discontinuities in skin (e.g., texture irregularities, fine lines, wrinkles, sagging, stretch marks, cellulite, puffy eyes, and the like in the skin which may be detected visually or by feel). As used herein, therapeutically regulating skin condition includes ameliorating (e.g., diminishing, minimizing and/or effacing, discontinuities in skin). Regulating skin condition involves improving skin appearance and/or feel. As used herein, “regulating skin condition” is intended to include regulation of such signs irrespective of the mechanism of origin.

A wide range of quantities of the compositions of the present invention can be employed to provide a skin, hair or nail appearance and/or feel benefit. Quantities of the present compositions, which are typically applied per application are, in milligrams of composition over a square centimeter of the skin herein referred to as “mg/cm²”, is from about 0.1 mg/cm² to about 20 mg/cm². A particularly useful application amount is about 0.5 mg/cm² to about 10 mg/cm².

Actives

An embodiment of the invention comprises a composition comprising at least one active ingredient. The active ingredient can be a skin care active. Among the active ingredients that can be selected are those listed below. Mixtures of those listed can also be used. The compositions may optionally comprise a safe and effective amount of an active. Examples of actives which can be included are listed below. These actives are preferably present at from about 0.0001% to about 20%, more preferably from about 0.05% to about 5%, even more preferably from about 0.1% to about 2%, by weight of the composition. Exceptions to these preferred ranges are those for conditioning agents and peptides which are specified below.

Actives can have more than one mechanism of action, so their placement in specific categories does not necessarily encompass their entire range of activity. Non-limiting examples of actives useful in the present invention include:

Actives

1. Desquamation Actives

An example of a desquamation active is salicylic acid. Other examples are \acute{α}-hydroxy acids (e.g. glycolic acid, lactic acid) and \acute{α}-keto acids (e.g. pyruvic acid).

2. Anti-Acne Actives

Examples of useful anti-acne actives include resorcinol, sulfur, erythromycin, zinc, and dehydroacetic acid. Further examples of suitable anti-acne actives are described in further detail in U.S. Pat. No. 5,607,980.

3. Anti-Wrinkle Actives/Anti-Atrophy Actives

Exemplary anti-wrinkle/anti-atrophy actives suitable for use in the compositions of the present invention include hydroxy acids (e.g., salicylic acid, glycolic acid), keto acids (e.g., pyruvic acid), ascorbic acid (vitamin C), phytic acid, lysophosphatidic acid, flavonoids (e.g., isoflavones, flavones, etc.), stilbenes, cinnamates, resveratrol, kinetin, zeatin, dimethylaminoethanol, synthetic peptides, peptides from natural sources (e.g., soy peptides), salts of sugar acids (e.g., Mn gluconate), retinoids (e.g. retinyl propionate), vitamin B compounds (e.g., thiamine (vitamin B1), niacinamide (vitamin B3), panthenol and pantothenic acid (vitamin B5), pyridoxine (vitamin B6), carnitine (vitamin Bt), riboflavin (vitamin B2), and their derivatives and salts (e.g., HCl salts or calcium salts)).

4. Anti-Oxidants/Radical Scavengers

Non-limiting examples of anti-oxidants/radical scavengers useful in the present invention include ascorbic acid (vitamin C) and its salts, ascorbyl esters of fatty acids, ascorbic acid derivatives (e.g., magnesium ascorbyl phosphate), tocopherol (vitamin E), tocopherol sorbate, tocopherol acetate, other esters of tocopherol, butylated hydroxy benzoic acids and their salts (e.g. butylated hydroxyl toluene which is commonly known as BHT), 6-hydroxy-2,5,7,8-tetramethylchroman-2-carboxylic acid, amines (e.g., N,N-diethylhydroxylamine, aminoguanidine), nordihydroguaiaretic acid, bioflavonoids, amino acids, silymarin, tea extracts, and grape skin/seed extracts. Preferred anti-oxidants/radical scavengers are selected from grape skin/seed extracts, tea extracts, and esters of tocopherol, more preferably tocopherol acetate.

5. Chelators

Exemplary chelators that are useful herein are disclosed in U.S. Pat. No. 5,487,884.

6. Flavonoids

Flavonoids are broadly disclosed in U.S. Pat. Nos. 5,686,082 and 5,686,367. Examples of flavonoids particularly suitable for use in the present invention are one or more flavones, one or more chalcones, one or more flavanones (e.g. hesperidin), one or more isoflavones, one or more coumarins, one or more chromones, one or more dicoumarols, one or more chromanones, one or more chromanols, isomers (e.g., cis/trans isomers) thereof, derivatives thereof, and mixtures thereof. Preferred for use herein are flavones and isoflavones, in particular daidzein (7,4′-dihydroxy isoflavone), genistein (5,7,4′-trihydroxy isoflavone), equol (7,4′-dihydroxy isoflavan), 5,7-dihydroxy-4′-methoxy isoflavone, soy isoflavones (a mixture extracted from soy), and mixtures thereof.

7. Anti-Inflammatory Agents

Steroidal anti-inflammatory agents include but are not limited to, corticosteroids such as hydrocortisone. A second class of anti-inflammatory agents, which is useful in the compositions, includes the nonsteroidal anti-inflammatory agents. The varieties of compounds encompassed by this group are well known to those skilled in the art. Specific non-steroidal anti-inflammatory agents useful in the composition invention include, but are not limited to, salicylates, flufenamic acid, etofenamate, aspirin, and mixtures thereof.

Additional anti-inflammatory agents useful herein include allantoin and compounds of the Licorice (the plant genus/species Glycyrrhiza glabra) family, including glycyrrhetic acid, glycyrrhizic acid, and derivatives thereof (e.g., salts and esters).

8. Anti-Cellulite Agents

The compositions of the present invention may optionally comprise a safe and effective amount of an anti-cellulite agent. Suitable agents may include, but are not limited to, xanthine compounds (e.g., caffeine, theophylline, theobromine, and aminophylline).

9. Tanning Actives

A preferred tanning active is dihydroxyacetone.

10. Skin Lightening Agents

Suitable skin lightening agents include those known in the art, including kojic acid, arbutin, tranexamic acid, ascorbic acid and derivatives thereof (e.g., magnesium ascorbyl phosphate or sodium ascorbyl phosphate, ascorbyl glucoside, and the like). Other skin lightening materials suitable for use herein include Actiwhite® (Cognis), Emblica ® (Rona), Azeloglicina (Sinerga), Sepiwhite, hexamidine, sugar amines, (e.g., N-acetyl glucosamine), phytosterols (e.g. one or more sitosterol, stigmasterol, campesterol, brassicasterol, etc.), and extracts (e.g. mulberry extract).

11. Antimicrobial and Antifungal Actives

Preferred examples of actives useful herein include those selected from the group consisting of salicylic acid, benzoyl peroxide, 3-hydroxy benzoic acid, glycolic acid, lactic acid, 4-hydroxy benzoic acid, acetyl salicylic acid, 2-hydroxybutanoic acid, 2-hydroxypentanoic acid, 2-hydroxyhexanoic acid, cis-retinoic acid, trans-retinoic acid, retinol, phytic acid, N-acetyl-L-cysteine, lipoic acid, azelaic acid, arachidonic acid, tetracycline, ibuprofen, naproxen, hydrocortisone, acetominophen, resorcinol, phenoxyethanol, phenoxypropanol, phenoxyisopropanol, 2,4,4′-trichloro-2′-hydroxy diphenyl ether, 3,4,4′-trichlorocarbanilide, octopirox, cyclopirox, lidocaine hydrochloride, clotrimazole, miconazole, ketoconazole, neomycin sulfate, and mixtures thereof.

12. Sunscreen Actives

Suitable sunscreen actives may be organic or inorganic. Sagarin, et al., at Chapter VIII, pages 189 et seq., of Cosmetics Science and Technology (1972), discloses numerous suitable actives. Additional actives can be found in “The European Union Cosmetics Directive 76/768/EEC, Annex VII. Non-limiting examples of suitable sunscreen agents include octinoxate, avobenzone, oxybenzone, octocrylene, octisalate, homosalate, meradimate, ensulizole, zinc oxide, titanium dioxide, and mixtures of these compounds.

13. Conditioning Agents

The compositions of the present invention may comprise a conditioning agent selected from the group consisting of humectants, moisturizers, hair conditioners, or skin conditioners, including emollients. A variety of these materials can be employed and each can be present at safe and effective levels, preferably from about 0.01% to about 100%, more preferably from about 0.1% to about 30%, and even more preferably from about 0.5% to about 25% by weight of the composition. These materials include, but are not limited to, guanidine; urea; glycolic acid and glycolate salts; lactic acid and lactate salts; aloe vera in any of its variety of forms (e.g., aloe vera gel); polyhydroxy compounds such as sorbitol, mannitol, glycerol, hexanetriol, butanetriol, propylene glycol, butylene glycol, hexylene glycol and the like; polyethylene glycols; sugars (e.g., melibiose) and starches; sugar and starch derivatives (e.g., alkoxylated glucose, fructose, sucrose, etc.); hyaluronic acid; panthenol and panthenol derivatives; lactamide monoethanolamine; acetamide monoethanolamine; sucrose polyester; petrolatum; silicones and silicone elastomers; hydrocarbon oils; fatty alcohols; fatty acids and esters; esters of mono and dibasic carboxylic acids with mono and polyhydric alcohols; polyolefins, polyoxyethylenes; polyoxypropylenes; mixtures of polyoxyethylene and polyoxypropylene ethers of fatty alcohols and mixtures thereof. Many conditioning agents are fatty materials which are soft and lubricious resulting in a material which not only conditions, but also is smooth in feel.

Silicones useful in the composition herein include polyalkylsiloxanes with viscosities of from about 0.5 to about 1,000,000 centistokes at 25° C. Additional silicones suitable for use herein are the silicone elastomers, including emulsifying or non-emulsifying crosslinked siloxane elastomers, silicone gums, silicone resins, amino silicones, cationic silicones, high refractive silicones or mixtures thereof. Emulsifying crosslinked organopolysiloxane elastomers can notably be chosen from the crosslinked polymers described in U.S. Pat. Nos. 5,412,004, 5,837,793, and 5,811,487.

Advantageously, the non-emulsifying elastomers are dimethicone/vinyl dimethicone crosspolymers. Such dimethicone/vinyl dimethicone crosspolymers are supplied by a variety of suppliers including Dow Corning (DC 9040, DC 9041, DC9045), General Electric (SFE 839), Shin Etsu (KSG-15, 16, 18), and Grant Industries (GRANSIL™ line of elastomers). Cross-linked organopolysiloxane elastomers useful in the present invention and processes for making them are further described in U.S. Pat. No. 4,970,252, U.S. Pat. No. 5,760,116, and U.S. Pat. No. 5,654,362. Additional crosslinked organopolysiloxane elastomers useful in the present invention are disclosed in Japanese Patent Application JP 61-18708, assigned to Pola Kasei Kogyo KK.

Preferably, the conditioning agent is selected from the group consisting of glycerol, urea, petrolatum, sucrose polyester, silicones, esters, and combinations thereof.

14. Vitamins

Examples of vitamins include, but are not limited to, water-soluble versions of vitamin B, vitamin B derivatives, vitamin C, vitamin C derivatives, vitamin K, vitamin K derivatives, vitamin D, vitamin D derivatives, vitamin E, vitamin E derivatives, and mixtures thereof. The vitamin compounds may be included as the substantially pure material, or as an extract obtained by suitable physical and/or chemical isolation from natural (e.g., plant) sources.

15. Particulate Materials

Nonlimiting examples of particulate materials useful in the present invention include colored and uncolored pigments, interference pigments, inorganic powders, organic powders, composite powders, optical brightener particles, exfoliants and combinations thereof. These particulates can be platelet shaped, spherical, elongated or needle-shaped, or irregularly shaped, surface coated or uncoated, porous or non-porous, charged or uncharged, and can be added to the current compositions as a powder or as a pre-dispersion.

Particulate materials useful herein include but are not limited to bismuth oxychloride, sericite, mica, mica treated with barium sulfate or other materials, zeolite, kaolin, silica, boron nitride, lauroyl lysine, nylon, polyethylene, talc, styrene, polypropylene, polystyrene, ethylene/acrylic acid copolymer, sericite, aluminum oxide, silicone resin, barium sulfate, calcium carbonate, cellulose acetate, PTFE, polymethyl methacrylate, starch, modified starches such as aluminum starch octenyl succinate, silk, glass, fibers, ground seeds, pumice, and mixtures thereof. Especially preferred are spherical powders with an average primary particle size from 0.1 to 75 microns, preferably from 0.2 to 30 microns.

Also useful herein are interference pigments. Interference pigments, for purposes of the present specification are defined as thin platelike layered particles having two or more layers of controlled thickness with different refractive indices that yield a characteristic reflected color from the interference of typically two, but occasionally more, light reflections, form different layers of the platelike particle. The most common examples of interference pigments are micas layered with about 50-300 nm films of TiO2, Fe2O3, silica, tin oxide, and/or Cr2O3. Such pigments are often pearlescent. Pearl pigments reflect, refract and transmit light because of the transparency of pigment particles and the large difference in the refractive index of mica platelets and, for example, the titanium dioxide coating. Useful interference pigments are available commercially from a wide variety of suppliers, for example, Rona (Timiron™ and Dichrona™), Eckart (e.g. Prestige and Prestige Silk lines). Especially preferred are interference pigments with smaller particle sizes, with an average diameter of individual particles less than about 75 microns in the longest direction, preferably with an average diameter less than about 50 microns.

Other pigments useful in the present invention provide color primarily through selective absorption of specific wavelengths of visible light, and include inorganic pigments, organic pigments and combinations thereof. Examples of useful inorganic pigments include iron oxides, ferric ammonium ferrocyanide, manganese violet, ultramarine blue, and Chrome oxide. Organic pigments can include natural colorants and synthetic monomeric and polymeric colorants. An example is phthalocyanine blue and green pigment. Also useful are lakes, primary FD&C or D&C lakes and blends thereof. Also useful are encapsulated soluble or insoluble dyes and other colorants. Inorganic white or uncolored pigments useful in the present invention, for example TiO2, ZnO, or ZrO2, are commercially available from a number of sources. One example of a suitable particulate material contains the material available from U.S. Cosmetics (TRONOX TiO2 series, SAT-T CR837, a rutile TiO2). Particularly preferred are charged dispersions of titanium dioxide, as are disclosed in U.S. Pat. No. 5,997,887.

The pigments/powders useful herein can be surface treated to provide added stability of color and/or for ease of formulation. Non-limiting examples of suitable coating materials include silicones, lecithin, amino acids, metal soaps, polyethylene and collagen. These surface treatments may be hydrophobic or hydrophilic, with hydrophobic treatments being preferred. Particularly useful hydrophobic pigment treatments include polysiloxane treatments such as those disclosed in U.S. Pat. No. 5,143,722.

16. Sugar Amines (Amino Sugars)

The sugar amine compounds useful in the present invention are described in PCT Publication WO 02/076423 and U.S. Pat. No. 6,159,485.

Sugar amines can be synthetic or natural in origin and can be used as pure compounds or mixtures of compounds (e.g., extracts from natural sources or mixtures of synthetic materials). Glucosamine is generally found in many shellfish and can also be derived from fungal sources. As used herein, “sugar amine” includes isomers and tautomers of such and its salts (e.g., HCl salt) and is commercially available from Sigma Chemical Co.

Examples of sugar amines that may be useful herein include glucosamine, N-acetyl glucosamine, mannosamine, N-acetyl mannosamine, galactosamine, N-acetyl galactosamine, their isomers (e.g., stereoisomers), and their salts (e.g., HCl salt). Preferred for use herein are glucosamine, particularly D-glucosamine and N-acetyl glucosamine, particularly N-acetyl-D-glucosamine

17. Vitamin B₃ Compounds

The compositions of the present invention may optionally comprise a safe and effective amount of a vitamin B₃ compound. Vitamin B₃ compounds are particularly useful for regulating skin conditions as described in U.S. Pat. No. 5,939,082.

Exemplary derivatives of the foregoing vitamin B₃ compounds include nicotinic acid esters, including non-vasodilating esters of nicotinic acid (e.g., tocopheryl nicotinate, myristyl nicotinate).

Examples of suitable vitamin B₃ compounds are well known in the art and are commercially available from a number of sources (e.g., the Sigma Chemical Company, ICN Biomedicals, Inc., and Aldrich Chemical Company).

18. Retinoids

As used herein, “retinoid” includes all natural and/or synthetic analogs of Vitamin A or retinol-like compounds which possess the biological activity of Vitamin A in the skin as well as the geometric isomers and stereoisomers of these compounds. The retinoid is preferably selected from retinol, retinol esters (e.g., C₂-C₂₂ alkyl esters of retinol, including retinyl palmitate, retinyl acetate, retinyl propionate), retinal, and/or retinoic acid (including all-trans retinoic acid and/or 13-cis-retinoic acid), or mixtures thereof. More preferably the retinoid is a retinoid other than retinoic acid. Preferred retinoids are retinol, retinyl palmitate, retinyl acetate, retinyl propionate, retinal and combinations thereof. More preferred is retinyl propionate.

19. Peptides and Amino Acids

The compositions of the present invention may optionally comprise a safe and effective amount of a peptide, including but not limited to, di-, tri-, tetra-, penta-, and hexa-peptides and derivatives thereof for regulating keratinous tissue. The compositions contain preferably from about 1×10⁻⁷% to about 20%, more preferably from about 1×10⁻⁴% to about 10%, even more preferably from about 1×10⁻⁵% to about 5%, by weight of the composition of peptide or amino acid.

As used herein, “peptide” refers to peptides containing ten or fewer amino acids and their derivatives, isomers, and complexes with other species such as metal ions (e.g., copper, zinc, manganese, magnesium, and the like). As used herein, peptide refers to both naturally occurring and synthesized peptides. Also useful herein are naturally occurring and commercially available compositions that contain peptides. More preferred peptides are the dipeptide carnosine (beta-ala-his), the tripeptide gly-his-lys, the pentapeptide lys-thr-thr-lys-ser, lipophilic derivatives of peptides, and metal complexes of the above, e.g., copper complex of the tripeptide his-gly-gly (also known as lamin). A preferred commercially available tripeptide derivative-containing composition is Biopeptide CL®, which contains 100 ppm of palmitoyl-gly-his-lys and is commercially available from Sederma. A preferred commercially available pentapeptide derivative-containing composition is Matrixyl®, which contains 100 ppm of palmitoyl-lys-thr-thr-lys-ser and is commercially available from Sederma.

Useful amino acids include but are not limited to aspargine, alanin, indole, glutamic acid, tyrosine, tryptamine and their salts and combinations thereof.

20. Phytosterols

Examples of suitable phytosterols include β-sitosterol, campesterol, brassicasterol, D5-avennasterol, lupenol, \acute{α}-spinasterol, stigmasterol, their derivatives, analogs, and combinations thereof. Preferably, the phytosterol is selected from the group consisting of β-sitosterol, campesterol, brassicasterol, stigmasterol, their derivatives, and combinations thereof. More preferably, the phytosterol is stigmasterol.

Phytosterols can be synthetic or natural in origin and can be used as essentially pure compounds or mixtures of compounds (e.g., extracts from natural sources). Phytosterols are generally found in the unsaponifiable portion of vegetable oils and fats and are available as free sterols, acetylated derivatives, sterol esters, ethoxylated or glycosidic derivatives. More preferably, the phytosterols are free sterols. As used herein, “phytosterol” includes isomers and tautomers of such and is commercially available from Aldrich Chemical Company, Sigma Chemical Company, and Cognis.

21. Hexamidines

The compositions of the present invention may optionally comprise a safe and effective amount of hexamidine compounds, their salts, and derivatives for regulating keratinous tissue.

As used herein, hexamidine derivatives include any isomers and tautomers of hexamidine compounds including but not limited to organic acids and mineral acids, for example sulfonic acid, carboxylic acid, etc. Preferably, the hexamidine compounds include hexamidine diisethionate, commercially available as Eleastab® HP100 from Laboratoires Serobiologiques.

22. Dialkanoyl Hydroxyproline Compounds

The compositions of the present invention may optionally comprise a safe and effective amount of one or more dialkanoyl hydroxyproline compounds and their salts and derivatives for regulating keratinous tissue.

Suitable derivatives include but are not limited to esters, for example fatty esters, including, but not limited to tripalmitoyl hydroxyproline and dipalmityl acetyl hydroxyproline. A particularly useful compound is dipalmitoyl hydroxyproline. As used herein, dipalmitoyl hydroxyproline includes any isomers and tautomers of such and is commercially available under the tradename Sepilift DPHP® from Seppic, Inc. Further discussion of dipalmitoyl hydroxyproline appears in PCT Publication WO 93/23028. Preferably, the dipalmitoyl hydroxyproline is the triethanolamine salt of dipalmitoyl hydroxyproline.

23. Salicylic Acid Compounds

The topical compositions of the present invention may optionally comprise a safe and effective amount of a salicylic acid compound, its esters, its salts, or combinations thereof for regulating keratinous tissue.

24. N-acyl Amino Acid Compounds

The topical compositions of the present invention may optionally comprise a safe and effective amount of one or more N-acyl amino acid compounds. The amino acid can be one of any of the amino acids known in the art. Preferably, the N-acyl amino acid compound is selected from the group consisting of N-acyl Phenylalanine, N-acyl Tyrosine, their isomers, their salts, and derivatives thereof. The amino acid can be the D or L isomer or a mixture thereof

Particularly useful as a topical skin tone evening cosmetic agent is N-undecylenoyl-L-phenylalanine. As used herein, N-undecylenoyl-L-phenylalanine is commercially available under the tradename Sepiwhite® from SEPPIC.

25. Dehydroacetic Acid (DHA)

The composition of this invention may optionally comprise a safe and effective amount of dehydroacetic acid or pharmaceutically acceptable salts, derivatives or tautomers thereof for regulating keratinous tissue. The technical name for dehydroacetic acid is 3-Acetyl-6-methyl-2H-pyran-2,4(3H)-dione. The compound can be commercially purchased from UniversalPreserv-A-Chem, Inc. as Unisept DHA.

Pharmaceutically acceptable salts include alkali metal salts, such as sodium and potassium; alkaline earth metal salts, such as calcium and magnesium; non-toxic heavy metal salts; ammonium salts; and trialkylammonium salts, such astrimethylammonium and triethylammonium. Sodium, potassium, and ammonium salts of dehydroacetic acid are preferred. Highly preferred is sodium dehydroacetate which can be purchased from Tri-K, as Tristat SDHA. Derivatives of dehydroacetic acid incude, but are not limited to, any compounds wherein the CH3 groups are individually or in combination replaced by amides, esters, amino groups, alkyls, and alcohol esters. Tautomers of dehydroacetic acid are the isomers of dehydroacetic acid which can change into one another with great ease so that they ordinarily exist in equilibrium. Thus, tautomers of dehydroacetic acid can be described as having the chemical formula C8H8O4 and generally having the structure above.

26. Skin Firming Agents:

Skin firming agents are materials that produce an immediate firming or tightening sensation shortly after application to the skin. Further, through this tightening or skin tensing effect, these materials may also provide a visible skin smoothing effect, reducing the appearance of skin texture, fine lines, and wrinkles.

Non-limiting examples of skin firming agents useful in the present invention include a variety of natural and synthetic polymers, such as those described in U.S. Pat. No. 6.284,233, which is included by reference herein. For example, protein extracts from wheat and peas, or egg albumin are effective skin firming agents. Other materials such as the silicates described in EP 1008340, also included by reference herein, are also suitable for use in the compositions of the present invention.

27. Anti-Dandruff Actives

Particulate, crystalline agents dispersed and suspended throughout the topical composition may provide anti-dandruff activity during a shampooing process.

Anti-dandruff actives used for this purpose include polyvalent metal salts of pyrithione, salicylic acid, coal tar, pine tar, sulfur, whitfield's ointment, castellani's paint, aluminum chloride, gentian violet, octopirox (piroctone olamine), ciclopirox olamine, undecylenic acid and it's metal salts, potassium permanganate, selenium sulphide, sodium thiosulfate, propylene glycol, oil of bitter orange, urea preparations, griseofulvin, 8-Hydroxyquinoline ciloquinol, thiobendazole, thiocarbamates, haloprogin, polyenes, hydroxypyridone, morpholine, benzylamine, allylamines (such as terbinafine), tea tree oil, extracts of melaleuca (tea tree), charcoal, clove leaf oil, coriander, palmarosa, berberine, thyme red, climbasole, innamon oil, cinnamic aldehyde, citronellic acid, hinokitol, ichthyol pale, Sensiva SC-50, Elestab HP-100, azelaic acid, lyticase, iodopropynyl butylcarbamate (IPBC), isothiazalinones such as octyl isothiazalinone and azoles, and combinations thereof.

Other Ingredients

The compositions of the present invention can contain a wide variety of ingredients that are used in conventional product types, provided that they do not unacceptably alter the benefits of the invention. Additionally, these ingredients, when incorporated into the composition, should be suitable for use in contact with mammalian keratinous tissue without undue toxicity, incompatibility, instability, allergic response, and the like within the scope of sound judgment. The CTFA Cosmetic Ingredient Handbook, Second Edition (1992) describes a wide variety of nonlimiting cosmetic and pharmaceutical ingredients commonly used in the care of keratinous tissue, which are suitable for use in the compositions of the present invention. Examples of these and similar ingredient classes include: abrasives, absorbents, aesthetic components such as fragrances, pigments, colorings/colorants, essential oils, skin sensates, astringents, etc. (e.g., clove oil, menthol, camphor, eucalyptus oil, eugenol, menthyl lactate, witch hazel distillate), anti-acne agents, anti-caking agents, antifoaming agents, antimicrobial agents (e.g., iodopropynyl butylcarbamate), antioxidants, binders, biological additives, buffering agents, bulking agents, chelating agents, chemical additives, colorants, cosmetic astringents, cosmetic biocides, denaturants, drug astringents, emollients, external analgesics, film formers or materials, e.g., polymers, for aiding the film-forming properties or substantivity of the composition (e.g., copolymer of eicosene and vinyl pyrrolidone), cationic polymers (for example, quaternary ammonium or cationic protonated amino moieties), nonionic polymers, humectants, opacifying agents, pH adjusters, propellants, reducing agents, sequestering agents, skin bleaching and lightening agents, hair and skin-conditioning agents, skin soothing and/or healing agents and derivatives, hair and skin treating agents, surfactants, thickeners, and vitamins and derivatives thereof. Additional examples of suitable emulsifiers and surfactants can be found in, for example, U.S. Pat. No. 3,755,560, U.S. Pat. No. 4,421,769, and McCutcheon's Detergents and Emulsifiers, North American Edition, pages 317-324 (1986). It should be noted, however, that many materials may provide more than one benefit, or operate via more than one mode of action. Therefore, classifications herein are made for the sake of convenience and are not intended to limit the active to that particular application or applications listed.

Composition Forms

The physical form of the topical compositions is not critical. The topical compositions of the present invention can be in any form known in the art, including but not limited to solutions, suspensions, dispersions, emulsions, and combinations of these.

For example, emulsion carriers, including but not limited to oil-in-water, water-in-oil, water-in-oil-in-water, and oil-in-water-in-oil emulsions are useful herein. In emulsion systems, as will be understood by the skilled artisan, a given component will distribute primarily into either the water or the oil phase, depending on water solubility/dispersibility of the component in the composition. Preferred emulsions are oil-in-water emulsions and water-in-oil emulsions, especially when all or a portion of the oil phase is a silicone or blend of silicones.

Water in oil emulsions may extend the duration of the perceived warmth of the composition when applied to the keratinous tissue. Not wishing to be bound by theory, it is believed that since water is in the discontinuous phase of the emulsion, it will evaporate more slowly, thus resulting in a slower cooling rate of the product film on the surface. This is due to the fact that evaporation is a cooling process. In one embodiment, the composition is an water in oil emulsion wherein the majority of the oil is silicone. This type of composition is often called water-in-silicone emulsions.

Non-limiting examples of emulsifiers useful in the emulsions of this invention are given in McCutcheon's, Detergents and Emulsifiers, North American Edition (1986), published by Allured Publishing Corporation; U.S. Pat. No. 5,011,681; U.S. Pat. No. 4,421,769; and U.S. Pat. No. 3,755,560. Exemplary carriers and such other ingredients that are suitable for use herein are described, for example, in U.S. Pat. No. 6,060,547.

The duration of the perceived warmth can also be extended by the inclusion of one or more waxy materials with melting points of about 30° C. to about 80° C. Such compositions contain greater than about 1%, more preferably greater than about 2.0%, even more preferably greater than about 2.5% by weight of the composition of one or a combination of such waxy materials. Not wishing to be bound by theory, these waxy materials melt when the composition is heated in the sachet and then, when applied to the keratinous tissue, the melted waxy materials re-crystallize releasing heat and thus reducing the cooling rate of the product on the surface.

As will be understood by one skilled in the art, the topical compositions of the present invention can exhibit a wide range of viscosities, depending on the type and level of thickening or structuring agent or agents included in the formulation. In particular, compositions can range from very thin and flowable (liquids, milks, lotions, serums, thin gels) to thicker products (creams, thicker gels, pastes, ointments) to even semi-solid and solid (powders, sticks).

Viscosity, as measured using commercially available viscometers/rheometers (for example, the Brookfield model series DV-II and DV-III viscometers from Brookfield Engineering Laboratories, Inc) is one known way to characterize the thickness of a composition. In one preferred embodiment of the current invention, the viscosity of the topical composition is between 10,000 cps and 500,000 cps when measured at the target temperature of the topical composition. In another preferred embodiment of the invention, the viscosity of the topical composition at it's target temperature is at least 50% of the viscosity of the topical composition at 22° C., even more preferably at least 80%. In this way, the composition does not become overly thin or messy during application, and its rheological properties will remain relatively constant regardless of whether a person uses the product at room temperature or at any temperature up to the target temperature of the heater system.

The topical compositions herein can be cleansers, including, but not limited to, shampoos, body washes, liquid soaps, and scrubs. The topical compositions can also be moisturizers; anti-aging products; sunscreens; pharmaceutical products; dermatological products; hair colorants; hair conditioners; antiperspirants; deodorants; toothpaste; cosmetics, including but not limited to foundation, blush mascara, lipstick and nail polish; depilatories; patches, masks; and massage products.

The topical composition may optionally comprise materials which change color or level of opacity when the topical composition has been warmed. This would result in a visual signal based in the topical composition.

Composition Preparation

The compositions of the present invention are generally prepared by conventional methods such as are known in the art of making topical compositions. Such methods typically involve mixing of the ingredients in one or more steps to a relatively uniform state, with or without heating, cooling, application of vacuum, and the like. The compositions are preferably prepared such as to optimize stability (physical stability, chemical stability, photostability) and/or delivery of the active materials. This optimization may include appropriate pH (e.g., less than 7), exclusion of materials that can complex with the active agent and thus negatively impact stability or delivery (e.g., exclusion of contaminating iron), use of approaches to prevent complex formation (e.g., appropriate dispersing agents or dual compartment packaging), use of appropriate photostability approaches (e.g., incorporation of sunscreen/sunblock, use of opaque packaging), etc.

### EXAMPLES

The following are non-limiting examples of the compositions of the present invention. The examples are given solely for the purpose of illustration and are not to be construed as limitations of the present invention, as many variations thereof are possible without departing from the spirit and scope of the invention, which would be recognized by one of ordinary skill in the art. In the examples, all concentrations are listed as weight percent, unless otherwise specified and may exclude minor materials such as diluents, filler, and so forth. The listed formulations, therefore, comprise the listed components and any minor materials associated with such components. As is apparent to one of ordinary skill in the art, the selection of these minors will vary depending on the physical and chemical characteristics of the particular ingredients selected to make the present invention as described herein.

### Examples 1-3

### Silicone-In-Water Emulsion Lotions/Serums

| | 1 | 2 | 3 |
|---|---|---|---|
| Water Phase: | | | |
| Water | QS to 100% | QS to 100% | QS to 100% |
| Glycerin | 5 | 10 | 7 |
| Butylene Glycol | 2 | — | — |
| Aloe Vera Gel | — | 0.1 | 0.1 |
| Grapeseed Extract | — | — | 0.01 |
| Green Tea Extract | — | 0.5 | 0.1 |
| Glydant Plus Liquid⁹ | 0.3 | 0.3 | 0.3 |
| N-Acetyl Glucosamine | — | 0.5 | 2 |
| Niacinamide | 3 | 5 | 4 |
| Dex-Panthenol | 0.5 | 1 | 0.5 |
| Disodium EDTA | 0.05 | 0.05 | 0.1 |
| Polysorbate 20 | 0.3 | 0.5 | — |
| Laureth-4 | 0.1 | — | 0.2 |
| Oil Phase: | | | |
| Cyclomethicone D5 | 10 | 14 | 10 |
| Dimethicone | — | — | 3 |
| Isohexadecane | 3 | — | — |
| Phenyl Trimethicone | — | 2 | — |
| Dow Corning 9045¹ | — | — | 8 |
| USG-103² | — | 4 | — |
| Dow Corning 1503³ | — | 0.5 | 2 |
| Thickener: | | | |
| Simulgel EG⁶ | 3 | — | — |
| Carbopol Ultrez 21⁷ | — | 0.35 | — |
| Sepigel 305⁸ | — | — | 2 |
| Additional Ingredients: | | | |
| Polymethylsilsesquioxane | — | 2 | — |
| Microthene FN510-00⁴ | — | — | 1 |
| Titanium Dioxide | 0.25 | — | — |
| Prestige Silk Red⁵ | — | — | 1 |
| Triethanolamine | — | 0.5 | — |
| Colored Dyes | 0.002 | — | — |
| Fragrance | 0.05 | — | 0.1 |

¹A silicone elastomer blend from Dow Corning Corporation
²A silicone elastomer blend from ShinEtsu
³A silicone gum blend from Dow Corning Corporation
⁴A spherical polyethylene powder from Equistar
⁵A layered mica/titanium dioxide/tin oxide interference pigment from Eckart
⁶A sodium acrylate/acryloyldimethyl taurate copolymer thickening agent from Seppic
⁷An acrylates/C10-30 alkyl acrylate crosspolymer from Noveon
⁸A polyacrylamde based copolymer (with some sulfonic acid functionality) from Seppic
⁹A liquid preservative blend from Lonza

In a suitable vessel, combine the Carbopol and water phase ingredients, and then blend until uniform. In a separate suitable vessel, combine the oil phase ingredients and blend until uniform. Next, add the oil phase to the water phase and mill the resulting emulsion (eg., with a rotor-stator mill). Add the non-Carbopol thickener and remaining additional ingredients to the emulsion and blend until uniform. Fill sachets suitable for use with the instant receptacle.

### Examples 4-8

### Oil-In-Water Lotions/Creams

| | 4 | 5 | 6 | 7 | 8 |
|---|---|---|---|---|---|
| Water Phase: | | | | | |
| Water | qs | qs | qs | qs | qs |
| Glycerin | 3 | 5 | 7 | 10 | 15 |
| Disodium EDTA | 0.1 | 0.1 | 0.05 | 0.1 | 0.1 |
| Methylparaben | 0.1 | 0.1 | 0.1 | 0.1 | 0.1 |
| Niacinamide | 2 | 0.5 | — | 3 | 5 |
| Triethanolamine | — | 0.25 | — | — | — |
| D-panthenol | 0.5 | 0.1 | — | 0.5 | 1.5 |
| Sodium Dehydroacetate | — | 0.1 | 0.5 | 0.1 | 0.5 |
| Benzyl alcohol | 0.25 | 0.25 | 0.25 | 0.25 | 0.25 |
| GLW75CAP-MP (75% aq. | — | 0.5 | 0.5 | — | — |
| TiO2 dispersion)¹ | | | | | |
| Hexamidine diisethionate | — | 0.1 | — | — | — |
| Palmitoyl-pentapeptide² | 0.0003 | — | 0.0001 | — | 0.0003 |
| N-acetyl glucosamine | 2 | 1 | 2 | 2 | 1 |
| Soy Isoflavone | 0.5 | — | — | — | — |
| Oil Phase: | | | | | |
| Salicylic Acid | — | — | 1.5 | — | — |
| Isohexadecane | 3 | 3 | 3 | 4 | 3 |
| PPG15 Stearyl Ether | — | — | 4 | — | — |
| Isopropyl Isostearate | 1 | 0.5 | 1.3 | 1.5 | 1.3 |
| Sucrose polyester | 0.7 | — | 0.7 | 1 | 0.7 |
| Dipalmitoylbydroxyproline | — | — | — | 1.0 | 13 |
| Undecylenoyl | — | 0.5 | — | — | — |
| Phenylalanine | | | | | |
| Phytosterol | — | — | 0.5 | — | 1.0 |
| Cetyl alcohol | 0.4 | 0.3 | 0.4 | 0.5 | 0.4 |
| Stearyl alcohol | 0.5 | 0.35 | 0.5 | 0.6 | 0.5 |
| Behenyl alcohol | 0.4 | 0.3 | 0.4 | 0.5 | 0.4 |
| PEG-100 stearate | 0.1 | 0.1 | 0.1 | 0.2 | 0.1 |
| Cetearyl glucoside | 0.1 | 0.1 | 0.1 | 0.25 | 0.1 |
| Thickener: | | | | | |
| Polyacrylamide/C13-14 | 1.5 | — | 2 | 2.5 | 2 |
| isoparaffin/laureth-7 | | | | | |
| Sodium acrylate/sodium | — | 3 | — | — | — |
| acryloyldimethyl taurate | | | | | |
| copolymer/isobexadecane/p | | | | | |
| olysorbate 80 | | | | | |
| Additional Ingredients: | | | | | |
| Dimethicone/dimethiconol | — | 1 | 2 | 0.5 | 2 |
| Polymethylsilsequioxane | — | — | 0.25 | — | 1 |
| Nylon-12 | — | 0.5 | — | — | — |
| Prestige Silk Violet³ | — | — | — | — | 1 |
| Timiron Splendid Red⁴ | — | 1.0 | — | 2 | — |

¹Available from Kobo products
²Palmitoyl-lysine-threonine-threonine-lysine-serine available from Sederma
³Titanium dioxide coated mica violet interference pigment available from Eckart
⁴Silica and titanium dioxide coated mica red interference pigment available from Rona

In a suitable vessel, combine the water phase ingredients and heat to 75° C. In a separate suitable vessel, combine the oil phase ingredients and heat to 75° C. Next, add the oil phase to the water phase and mill the resulting emulsion (eg., with a rotor-stator mill). Then, add the thickener to the emulsion and cool the emulsion to 45° C. while stirring. At 45° C., add the remaining ingredients. Cool the product and stir to 30° C. Fill sachets suitable for use with the instant receptacle.

### Examples 9-13

### Silicone-In-Water Serums/Lotions

| | 9 | 10 | 11 | 12 | 13 |
|---|---|---|---|---|---|
| Water Phase: | | | | | |
| Water | qs | qs | qs | qs | qs |
| Glycerin | 3 | 5 | 10 | 15 | 10 |
| Disodium EDTA | 0.1 | 0.1 | 0.1 | 0.1 | 0.1 |
| Niacinamide | 2 | 0.5 | 3 | 5 | 3 |
| Sodium Dehydroacetate | 0.5 | 0.1 | 0.1 | 0.5 | 0.1 |
| D-panthenol | 0.5 | 0.1 | 0.5 | 1.5 | 0.5 |
| GLW75CAP-MP (75% aq. TiO2 | — | 0.4 | — | — | 0.4 |
| dispersion)¹ | | | | | |
| Ascorbyl Glucoside | — | — | — | — | 1 |
| Palmitoyl pentapeptide² | — | — | — | 0.0003 | — |
| Soy Isoflavone | — | 1 | — | — | — |
| N-acetyl glucosamine | 2 | — | — | 5 | — |
| Silicone/Oil Phase: | | | | | |
| Cyclomethicone D5 | 10 | 5 | 10 | 7.5 | 10 |
| Dow Corning 9040 | — | 10 | 5 | 7.5 | 5 |
| silicone elastomer³ | | | | | |
| KSG-15AP silicone Elastomer⁴ | 5 | 13 | 5 | 7.5 | 5 |
| Dimethione/ | — | 2 | 1 | 2 | 2 |
| dimethiconol | | | | | |
| Dimethicone 50 csk | 1 | — | — | — | — |
| Salicylic Acid | — | — | — | — | — |
| Phytosterol | — | — | 1.0 | — | 0.1 |
| PPG-15 Stearyl Ether | — | — | 4 | — | — |
| BHT | — | 0.5 | — | — | — |
| Vitamin E Acetate | — | 0.5 | 0.1 | — | 0.1 |
| Thickener: | | | | | |
| Polyacrylamide/C13-14 | 2.5 | 2.5 | — | — | — |
| isoparaffin/laureth-7 | | | | | |
| Sodiumacrylate/sodium acryloyl | — | — | 3 | — | — |
| dimethyl taurate | | | | | |
| copolymer/isohexadecane/ | | | | | |
| polysorbate 80 | | | | | |
| Acrylates/C10-30 alkyl acrylates | — | — | — | 0.7 | 0.5 |
| crosspolymer | | | | | |
| Undecylenoyl Phenylalanine | | | | | |
| Premix: | | | | | |
| Undecylenoyl Phenylalanine | — | — | — | 1 | — |
| Water | — | — | — | 24 | — |
| Triethanolamine | — | — | — | 0.5 | — |
| Dipalmitoyl Hydroxy-Proline | | | | | |
| Premix: | | | | | |
| Water | — | — | — | — | 4.4 |
| Triethanolamine | — | — | — | — | 0.1 |
| Dipalmitoylhyroxyproline | — | — | — | — | 1.0 |
| Additional Ingredients: | | | | | |
| Triethanolamine | — | — | — | 0.8 | 0.6 |
| Polymethylsilsequioxane | 0.5 | 0.5 | 1 | 1 | 0.5 |
| Polyethylene | — | 0.5 | 1.0 | — | — |
| Silca | — | — | 0.5 | — | — |
| Prestige Silk Red⁵ | — | — | 1.0 | 1.0 | 1.0 |

¹GLW75CAP-MP, 75% aqueous titanium dioxide dispersion from Kobo
²Palmitoyl-lysine-threonine-threonine-lysine-serine available from Sederma
³A silicone elastomer dispersion from Dow Corning Corp
⁴A silicone elastomer dispersion from Shin Etsu,
⁵Titanium dioxide coated mica red interference pigment from Eckart

In a suitable vessel, combine the water phase ingredients and mix until uniform. In a separate suitable container, combine the silicone/oil phase ingredients and mix until uniform. Separately, prepare the dipalmitoyl hydroxyproline premix and/or undecylenoyl phenylalanine premix by combining the premix ingredients in a suitable container, heating to about 70° C. while stirring, and cooling to room temperature while stirring. Add half the thickener and then the silicone/oil phase to the water phase and mill the resulting emulsion (eg., with a rotor-stator mill). If applicable, add the remainder of the thickener, the dipalmitoyl hydroxyproline premix and/or undecylenoyl phenylalanine premix, and then the remaining ingredients to the emulsion while stirring. Stir until the composition is uniform. Fill sachets suitable for use with the instant receptacle.

### Examples 14-19

### Moisturizing Water-In-Silicone Creams/Lotions

| Component | 14 | 15 | 16 | 17 | 18 | 19 |
|---|---|---|---|---|---|---|
| Phase A | | | | | | |
| water | qs | qs | qs | qs | qs | qs |
| allantoin | 0.2 | 0.2 | 0.2 | 0.2 | 0.2 | 0.2 |
| disodium EDTA | 0.1 | 0.1 | 0.1 | 0.1 | 0.1 | 0.1 |
| ethyl paraben | 0.2 | 0.2 | 0.2 | 0.2 | 0.2 | 0.2 |
| propyl paraben | 0.1 | 0.1 | 0.1 | 0.1 | 0.1 | 0.1 |
| Caffeine | — | 1 | — | — | — | 1 |
| BHT | — | 0.1 | — | 0.015 | — | — |
| dexpanthenol | 1 | 0.5 | 1 | 1 | 1 | 1 |
| glycerin | 7.5 | 10 | 15 | 7.5 | 5 | 15 |
| hexamidine isethionate | — | — | 0.1 | 0.5 | — | — |
| niacinamide | 2 | — | — | 2 | 3.5 | 5 |
| palmitoyl-pentapeptide¹ | — | — | — | — | 0.0003 | — |
| Phenylbenzimidazole sulfonic | — | — | — | — | 1 | — |
| acid | | | | | | |
| Sodium Dehydroacetate | 0.5 | — | — | 0.1 | 0.5 | 0.5 |
| benzyl alcohol | 0.25 | 0.25 | 0.25 | 0.25 | 0.25 | 0.25 |
| triethanolamine | — | — | — | — | 0.6 | — |
| green tea extract | 1 | 1 | 1 | 1 | 1 | 1 |
| Soy Isoflavone | — | 0.5 | — | — | — | — |
| N-acetyl glucosamine | 5 | — | 2 | 5 | 2 | — |
| sodium metabisulfite | 0.1 | 0.1 | 0.1 | 0.1 | 0.1 | 0.1 |
| Phase B | | | | | | |
| cyclopentasiloxane | 15 | 15 | 18 | 15 | 15 | 18 |
| titanium dioxide | 0.5 | 0.5 | 0.75 | 0.5 | 0.5 | 0.75 |
| Phase C | | | | | | |
| C12-C15 alkyl benzoate | — | — | — | 1.5 | 1.5 | — |
| vitamin E acetate | 0.5 | — | 1 | 0.5 | 0.5 | 1 |
| retinyl propionate | 0.3 | — | — | 0.2 | 0.2 | — |
| Undecylenoyl Phenylalanine | — | — | 0.5 | — | — | — |
| Dipalmitoyl hydroxyproline | — | 1 | — | — | — | — |
| Salicylic Acid | — | 1.5 | 1.5 | — | — | — |
| PPG-15 Stearyl Ether | 4 | 4 | 4 | — | — | — |
| Dehydroacetic Acid | — | 0.5 | 0.1 | — | — | — |
| phytosterol | 1 | 0.5 | — | — | — | — |
| Phase D | | | | | | |
| KSG-21 silicone elastomer² | 4 | 4 | 5 | 4 | 4 | 5 |
| Dow Corning 9040 silicone | 15 | 15 | 12 | 15 | 15 | 12 |
| elastomer³ | | | | | | |
| Abil EM-97 Dimethicone | 0.5 | — | — | 0.5 | 0.5 | — |
| Copolyol⁴ | | | | | | |
| polymethylsilsesquioxane | 2.5 | 2.5 | 2 | 2.5 | 2.5 | 2 |
| Undecylenoyl Phenylalanine Premix | | | | | | |
| Undecylenoyl Phenylalanine | — | — | — | — | 1 | — |
| Water | — | — | — | — | 24 | — |
| Triethanolamine | — | — | — | — | 0.5 | — |
| Phase E | | | | | | |
| Water | 8.8 | — | — | — | — | 8.85 |
| Triethanolamine | 0.2 | — | — | — | — | 0.25 |
| Dipalmitoylhyroxyproline | 0.5 | — | — | — | — | 1 |

¹Palmitoyl-lysine-threonine-threonine-lysine-serine available from Sederma
²KSG-21 is an emulsifying silicone elastomer available from Shin Etsu
³A silicone elastomer dispersion from Dow Corning Corp
⁴Abil EM-97 available from Goldschmidt Chemical Corporation

In a suitable vessel, blend the Phase A components together with a suitable mixer (e.g., Tekmar model RW20DZM) and mix until all of the components are dissolved. Then, blend the Phase B components together in a suitable vessel and mill using a suitable mill (e.g., Tekmar RW-20) for about 5 minutes. Add the Phase C components to the Phase B mixture with mixing. Then, add the Phase D components to the mixture of Phases B and C and then mix the resulting combination of Phase B, C and D components using a suitable mixer (e.g., Tekmar RW-20) for about 1 hour. If applicable, prepare the undecylenoyl phenylalanine premix and/or Phase E by combining all ingredients, heating the ingredients to 70° C. while stirring, and cooling back to room temperature while stirring. Add the undecylenoyl phenylalanine premix and/or Phase E to Phase A while mixing. Next, slowly add Phase A to the mixture of Phases B, C and D with mixing. Mix the resulting mixture continually until the product is uniform. Mill the resulting product for about 5 minutes using an appropriate mill (e.g., Tekmar T-25). Fill sachets suitable for use with the instant receptacle.

### Examples 20-23

### Water Based Solid Formulations

| | 20 | 21 | 22 | 23 |
|---|---|---|---|---|
| Water | qs | qs | qs | 95 |
| Propylene Glycol | 15 | 30 | 20 | 20 |
| Dipropylene Glycol | 40 | 30 | 45 | 40 |
| Sodium Stearate | 6 | 6 | 6 | 6 |
| Triethanolamine | 0.2 | 0.25 | — | — |
| N-Acetyl-D-Glucosamine | — | 2.0 | 0.5 | — |
| Undecyenoyl Phenylalanine | — | 0.5 | — | — |
| Niacinamide | 2 | — | 3.5 | — |
| Sodium Dehydroacetate | 0.5 | 0.5 | 0.1 | — |
| Dipalmitoyl Hydroxyproline | 1 | — | — | — |
| Triclosan | — | — | — | 0.2 |

Combine all ingredients into an appropriate size container, heat to 85° C., cool. Fill sachets suitable for use with the instant receptacle at approximately 65-70° C.

### Examples 24-27

### Anhydrous Solid Formulations

| | 24 | 25 | 26 | 27 |
|---|---|---|---|---|
| Isopropyl Isostearate | 10 | 10 | 5 | — |
| Octylmethoxycinnamate | 5 | — | 7.5 | — |
| Cyclomethicone | qs | qs | qs | qs |
| Phenyl trimethicone | — | 5 | 5 | 5 |
| Stearyl Alcohol | 15 | 17 | 15 | 15 |
| Behenyl Alcohol | 1 | 1 | 1 | 1 |
| Undecylenoyl Phenyl alanine | — | 0.5 | — | — |
| Dehydroacetic acid | 0.1 | 0.5 | 0.1 | — |
| Dipalmitoyl Hydroxyproline | 1 | — | 1.0 | — |
| Phytosterol | 1 | 0.5 | — | — |
| Salicylic Acid | — | — | 0.5 | — |
| Aluminum Zirconium | — | — | — | 20 |
| Trichlorohydrex Gly | | | | |

Add all ingredients to an appropriate size container, heat to 75° C. then cool with stirring until mixture reaches approximately 45-50° C. Fill sachets suitable for use with the instant receptacle.

### Examples 28-29

### Aqueous Gel Formulations

| | 28 | 29 |
|---|---|---|
| Water | qs | qs |
| Glycerin | 10 | 5 |
| Niacinamide | 4 | 2 |
| Sodium Dehydroacetate | — | 0.1 |
| N-Acel-D-Glucosamine | 2 | — |
| Glydant Plus Liquid | 0.3 | 0.3 |
| Disodium EDTA | 0.02 | 0.02 |
| Simulgel EG | 2.5 | — |
| Sepigel 305 | — | 3.0 |

Combine all ingredients into an appropriate size container and mix until uniform. Fill sachets suitable for use with the instant receptacle.

### Example 30

### Shampoo Formulation

| | |
|---|---|
| Sodium Laureth Sulfate(2 moles ethoxylate/29% active) | 34.5 |
| Sodium Lauryl Sulfate (29% active) | 13.8 |
| Cocoamidopropyl Betaine (30% active) | 6.7 |
| Cocoamide MEA | 0.5 |
| Guar (Jaquar C14 from Rhodia) | 0.1 |
| Perfume | 1.0 |
| Water | q.s. |

Combine all ingredients into an appropriate size container and mix until uniform. Fill sachets suitable for use with the instant receptacle.

### Example 31

### Facial Cleanser

| Water | q.s. |
|---|---|
| Cetyl Betaine | 2.00 |
| Sodium Alkyl Sulfate | 1.00 |
| PPG-14 Butyl Ether | 3.25 |
| Glycerin | 3.00 |
| Stearyl Alcohol | 2.88 |
| Polyethylene Particles¹ | 2.00 |
| Polyethylene Particles² | 2.00 |
| Salicylic Acid | 2.00 |
| Distearyl Dimethyl Ammonium Chloride | 1.50 |
| Cetyl Alcohol | 0.80 |
| Urea | 0.50 |
| Steareth-21 | 0.50 |
| Behenyl Alcohol | 0.32 |
| PPG-30 | 0.25 |
| Steareth-2 | 0.25 |
| Fragrance | 0.15 |
| Polysaccharide Gum | 0.15 |
| Disodium EDTA | 0.01 |

¹Oxidized Polyethylene Particles having a mean particle size diameter of 25 microns, available as Acumist A-25 from Allied Signal Corp.
²Oxidized Polyethylene Particles having a mean particle size diameter of 45 microns, available as Acumist A-45 from Allied Signal Corp.

Mix the water, glycerin, and disodium EDTA in a suitable vessel and heat to 75-80° C. with stirring. Heat the PPG-14 butyl ether, and the salicylic acid in a separate vessel to 75-80° C. with stirring to form an oil phase. Next add the stearyl alcohol, cetyl alcohol, and the behenyl alcohol to this oil phase while continuing to heat with stirring. Next add the distearyl dimethyl ammonium chloride, the steareth-2, and steareth-21, to the oil phase while still continuing to heat and stir. Then emulsify this oil phase into the water-containing mixture using a homogenizing mill. Cool the resulting emulsion with stirring to 45° C. and add the urea and fragrance. Cool the emulsion to room temperature with stirring. Then mix in the sodium alkyl sulfate and the cetyl betaine, followed by the polyethylene particles.

### Example 31

### Hair Colorant

| Dye | |
|---|---|
| Cocamidopropyl betaine | 7.00 |
| Oleic acid | 5.00 |
| Octylpolyglycoside | 5.00 |
| Ammonium hydroxide | 5.00 |
| Aminoethylpropanol | 0.10 |
| Sodium carbonate | 0.10 |
| Sodium bicarbonate | 0.50 |
| 1-acetoxy-2-methyl-naphthalene | 1.20 |
| 4-amino-2-hydroxytoluene | 0.60 |
| p-aminophenol | 1.00 |
| N,N-bis(2-hydroxyethyl)-p-phenylenediamine | 0.50 |
| Water | q.s. |
| DEVELOPER | |
| Hydrogen peroxide | 3.00 |
| Acrylates Copolymer | 1.50 |
| Acrylates/steareth-20 | 1.50 |
| EDTA | 0.10 |
| Phosphoric acid | 0.10 |
| Water | 93.80 |

### Example 32

### Foaming Face Wash

| Phase A | |
|---|---|
| Water | q.s. |
| Polyquaternium - 10¹ | 0.50 |
| Phase B | |
| Amphoteric Surfactant I² | 15.1 |
| Amphoteric Surfactant II³ | 4.0 |
| Phase C | |
| Glycerin | 3.0 |
| Polyol Alkoxy Ester⁴ | 1.6 |
| PFG-120 Methyl Glucose Dioleate⁵ | 0.6 |
| Phase D | |
| Preservative (phenoxyethanol) | 0.4 |
| Phase E | |
| Water | 1.0 |
| Na₄ EDTA | 0.1 |

¹Available as Polymer JR 400
²Na Lauriminodipropionate available as Mirataine H2CHA
³Cocoamphocarboxyglycinate (and) Na Lauryl Sulfate (and) Hexylene Glycol available as Miranol 2MCA MOD
⁴Available as Crothix
⁵Available as Glucamate DOE120

Heat the water to 65° C. Add the polyquaternium-10 to the water to form Phase A. Add the Phase B ingredients sequentially to this phase. Separately heat, the Phase C components to 65° C. Combine Phase C with this mixture. Then cool Phases A, B, and C to 40° C. Add Phase D and Phase E to this mix to form the resultant cosmetic composition.

### Examples 33-34

### Liquid Foundation Compositions

| | 32 | 33 |
|---|---|---|
| Phase A | | |
| Cyclopentasiloxane & Dimethicone | 8.000 | 5.600 |
| Copolyol | | |
| Silicone modified Acryl Resin¹ | 14.820 | 5.390 |
| Polyacrylates-g-Polysiloxane | — | 14.274 |
| Resin² | | |
| Tridecyl Neopentanoate³ | 6.000 | 4.200 |
| Cetyl Dimethicone Copolyol⁴ | 2.000 | 1.400 |
| Cyclohexasiloxane | 2.000 | — |
| Cyclopentasiloxane | 3.275 | 7.797 |
| Phase B | | |
| Ethoxylated C10-16 Alcohols⁵ | 0.500 | 0.500 |
| Propylparaben | 0.150 | 0.150 |
| Phase C | | |
| Yellow Pigment | 0.737 | 0.595 |
| Red Pigment | 0.238 | 0.195 |
| Black Pigment | 0.130 | 0.106 |
| Micronized Titanium Dioxide | 0.250 | 0.175 |
| Boron Nitride Treated Starch⁶ | 3.500 | 2.450 |
| Titanium Dioxide | 8.250 | 5.775 |
| Polymethylsilesquioxane⁷ | 1.500 | 1.050 |
| Hexamethylene Diisothionate/ | 1.500 | 1.050 |
| Polypropylene/Polycaprolactone | | |
| Crosspolymer (and) Silica⁸ | | |
| Dimethicone/vinyldimethicone | 2.000 | 1.050 |
| crosspolymer⁹ | | |
| Mixed Cyclomethicones and Epoxy | — | 30.000 |
| Gel¹⁰ | | |
| Phase D | | |
| Deionized Water | 40.000 | 15.583 |
| Silica Shells¹¹ | 2.500 | 0.700 |
| Phenoxyethanol | 0.250 | 0.250 |
| Trisodium EDTA | 0.100 | 0.100 |
| Sodium Chloride | 2.000 | 1.400 |
| Sodium Dehydroacetate | 0.300 | 0.210 |
| Monohydrate | | |
| Totals | 100.000 | 100.000 |

¹Shin-Etsu - KP-545
²3M - SA-70
³ISP - Ceraphyl 55
⁴DeGussa Goldsmith - Abil WE-09
⁵Rhodia - Rhodasurf L-7/90
⁶National Starch - Dry Flo Elite BN
⁷GE - Tospearl 145A
⁸Kobo - BPD-500T
⁹Dow Corning - DC 9506 Powder
¹⁰GE-Velvesil-1111-19-372
¹¹Kobo - Silica Shells

Combine deionized water and Silica Shells of Phase D and mix with propeller or dispersator until homogeneity is achieved. Add remaining Phase D ingredients and continue propeller or dispersator blending. Combine Phase A ingredients in jacketed vessel and begin mixing with rotor stator mill. Recirculate cold water through jacketed vessel. Blend Phase B ingredients together for ten minutes. Add blended Phase B into Phase A ingredients. Add Phase C Ingredients into Phase AB. Shear Phase ABC until it is completely deagglomerated and pigments have been reduced to their primary particle size. Emulsify Phase D into Phase ABC under moderate shear. Blend Phase ABCD with sweep wall mixing until uniform. Fill sachets suitable for use with the instant receptacle.

### Example 35

### Disposable Wipe

Prepare an emulsion (Composition A) containing about 96.4% water, 2% silicone oil, 0.15% carboxylic acid polymeric emulsifier, 1% water soluble alkylene polyol, 0.1% chelating agent, 0.2% anti-microbial agent, 0.15% organic base pH-adjusting agent, and fragrance in the following manner.

Use a standard size, 55 gallon, covered cylindrical drum fitted with a single shaft having dual propeller blades, driven by a motor rated at 1750 rpm, to prepare the emulsion. The shaft extends substantially through the depth of the drum (about 4 feet) and has a first blade that is positioned near the bottom of the drum and a second shaft positioned about 1 foot above the first blade. The first blade has a diameter approaching that of the drum diameter (about 2 feet), and the second blade has a diameter of about 1 foot.

Charge the drum with 40 to 48 gallons of tap water. Begin agitation of the water by operating the motor at its maximum rated speed. Continue agitation at this speed throughout the entire process.

Slowly add 0.52 lbs. Pemulen® TR-2 to allow mixing of the emulsifier with the water sufficient to disperse or avoid the formation of lumps of the emulsifier. Then add the following components in the order stated: 0.35 lbs tetrasodium EDTA; 3.49 lbs propylene glycol; 6.97 lbs dimethicone (Dow Corning 200 Fluid, 350 cs); 0.70 lbs Glydant Plus®; 0.01 lbs of fragrance. Mix each of the foregoing ingredients individually into the water until they are well blended into the resultant mixture, typically by agitating for a period of about 1 minute between addition of the individual ingredients, and for a period of about 5-10 minutes once all the ingredients are in the vessel. Then add 0.52 lbs triethanolamine and continue agitation for a period of about 15-20 minutes, or until the mixture is well blended.

Laminate together a wipe composed of a two-ply cellulose substrate of 100% NSK fiber, having a Basis Weight of 26 lbs per ply and a Caliper of 20 mils per ply, under pressure with an adhesive, and roll into a continuous web roll. Suitable adhesives have a wet strength that is sufficient for the plies to remain substantially bonded in use. Slit the web roll to achieve the desired finished wipe width (e.g., 11.5″ in the unfolded configuration), and z-fold so that the machine direction edges overlap about 0.5″.

Then impregnate the resultant slitted and folded webs with composition A by passing the individual webs over the top of individual manifolds having holes through which the composition is pumped under pressure onto the moving web. Pump the composition so as to provide a loading of about 15.5 grams of composition per finished wipe.

Following impregnation, cut the webs to the desired finished wipe length (e.g., about 8.5″). Insert wipes into sachets suitable for use with the instant receptacle.

Sachets

The sachets utilized herein comprise a plurality of surfaces defining at least one chamber for containing the composition. Thus while sachets having only one chamber are frequently used, a plurality of chambers or multi-chambered sachets can also be used in the present invention. Typically, one or more of the sachet surfaces has low thermal resistance. As more than one wall of the receptacle can have an associated heating system, sachets having a variety surfaces with varying thermal resistance will heat quickly and evenly.

The sachet may be manufactured utilizing a wide variety of materials alone or in combination. The surfaces of the sachet can be thick or thin. Materials can also be chosen to provide the desired thermal resistance, resistance to light, oxygen barrier, and moisture barrier. In preferred embodiments the sachet, in whole or in part comprises films, formed from plastics, polymers, and foils, in single layers, multiple layers, blended or metalized and combinations thereof. Examples of these materials are polyolefins, polyesters, nylon, polypropylene, polyethylene, ethylene vinyl acetate, metalized PET(polyethylene terephthalate), aluminum foil, EVOH (ethylene-vinyl alcohol copolymer), PVDC (polyvinylidene chloride), etc. Other materials can be used including but not limited to cardboard. An example of a material used for the sachet is a co-extruded film polypropylene on the outside and polyethylene/ethylene vinyl acetate blend on the inside as the sealant layer. Similar sachets are available from Flexpaq, South Plainfield, N.J.

Various means of opening a sachet are known in the art and can be used with the sachets disclosed herein. These include, but are not limited to peeling by means of a pull tab, tearing along a line of weakness, squeezing etc. Resealable sachets are also contemplated.

In some embodiments, the sachet can be designed to burst or rupture to release the topical composition contained within the sachet at a comparatively low force when desired by the consumer. This may be accomplished by having the sachet be a sealed pouch with both permanent seals and also seals that are “frangible”, i.e., rupturable. When the sachet is squeezed, the frangible seal will yield or fail first since it has a lower peel force to break the seal apart than the permanent seals. Adding stress concentrators in the seal geometry that will localize forces at a particular location can optimize the location of rupture. For example, pressurizing a pouch-form sachet having a V-notch seal will localize forces first at the apex of the V, causing that region to rupture first. Additionally, other seal angles and geometries of the seal can also be used to tailor dispensing forces for removal of topical composition from a sachet for particular applications. Additional discussion of rupturable seals, including their manufacture appears in U.S. Pat. No. 6,508,602.

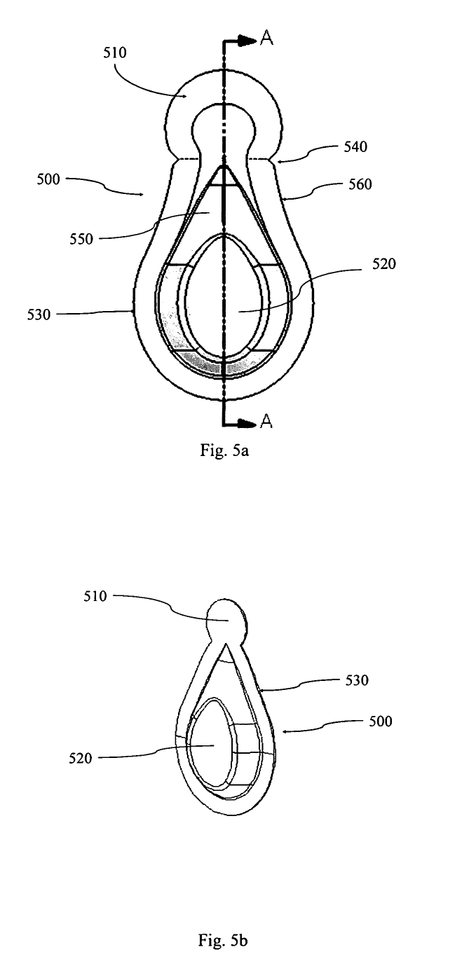

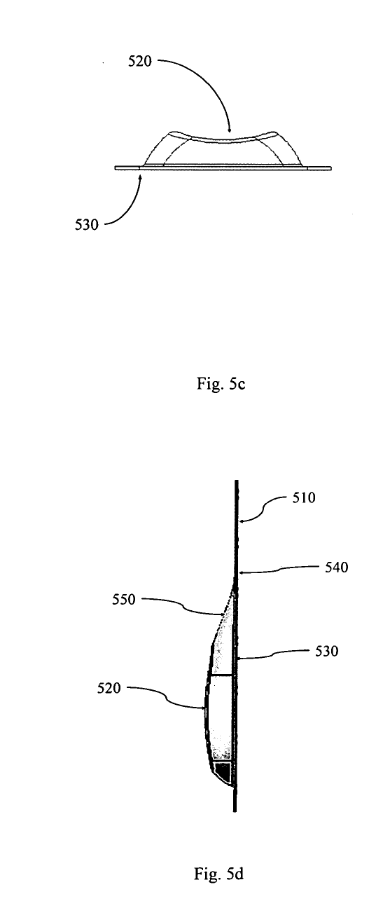

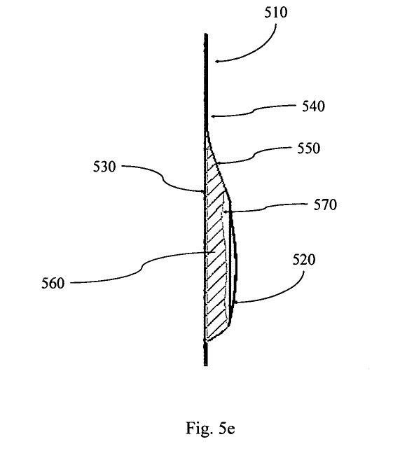

The embodiment of the sachet shown in FIG. 5a is a thermoformed blister pack 500 with a tear-off tab 510 that allows the topical composition to be dispensed when the tear-off tab 510 is removed along a line of weakness 540. This style of sachet is made by thermoforming, from a sheet of material, a front surface 550 of the sachet such that it forms a small chamber 520 to contain the topical composition. The chamber 520 is then filled and another layer of material, which forms the rear surface 530, is then sealed around the perimeter 560 of the chamber to contain the topical composition until ready to be used. FIGS. 5b through 5d provide further views of this embodiment. FIG. 5e illustrates a cross-sectional view of the sachet of FIG. 5a taken along line A-A, showing the topical composition 560 within the chamber 520. Other means of making sachets, pouches, packets, blisters, cells, thermoformed cups, sachets, and other sachet forms to contain a liquid, gel or solid are known in the art and can be used to make suitable reservoirs for this application.

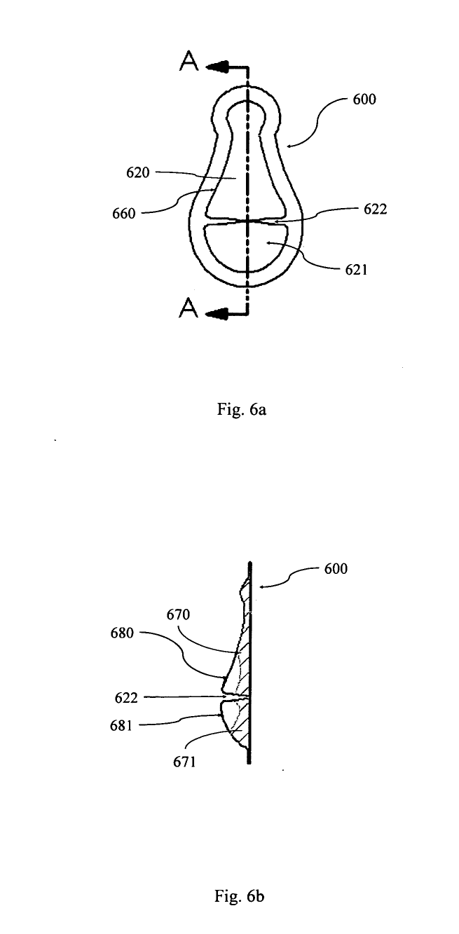

A sachet can comprise a plurality of compartments. Such “dual” or multiple compartment sachets are useful in a number of applications, especially when two incompatible products are to be applied. FIG. 6a depicts another embodiment of the sachet 600 that is functionally similar to that of FIG. 5a, but includes a plurality of chambers 620 and 621 separated by seal 622. FIG. 6b is a cross sectional view of the sachet shown in FIG. 6a taken along line A-A for containing topical compositions 670 and 671 and including the respective headspaces 680 and 681, shown in FIG. 6b. Respective chambers 620 and 621 may include topical composition(s) that are the same, similar, or diverse. The seal 622 separating the chambers 620 and 621 and the perimeter seal 660 may be designed to be ruptured sequentially or simultaneously depending on the peel force and on how pressure or squeezing is applied by the user.

Sachets, such as the sachet shown in FIG. 6a, having multiple chambers can also be designed for mixing incompatible topical compositions. This would allow the sachet to deliver multiple benefits such as cleansing from one chamber and moisturizing from another. The chamber containing the cleanser would be dispensed first to allow cleansing of the hair, nails, face, hands, or body and the second chamber containing the moisturizer is dispensed next to allow deep moisturizing of the hair, nails, face, hands, or body.

Alternatively a reaction such as foaming or additional self-heating could be accomplished by dispensing two topical compositions from the sachet that will react when mixed during dispensing. For instance, a dual compartment sachet could have one chamber containing sodium bi-carbonate and another chamber containing a water based cleanser such that when the two components meet they create a foaming topical composition.

Alternative to a sachet containing incompatible products, the sachets may also contain a singular product that due to instability of the components upon mixing requires combining its components just prior to application. In such examples, these components can be separated into a plurality of the sachet's chambers. Rupturable seals that separate these chambers can be burst by the user, allowing the composition to be freshly mixed just prior to their application.

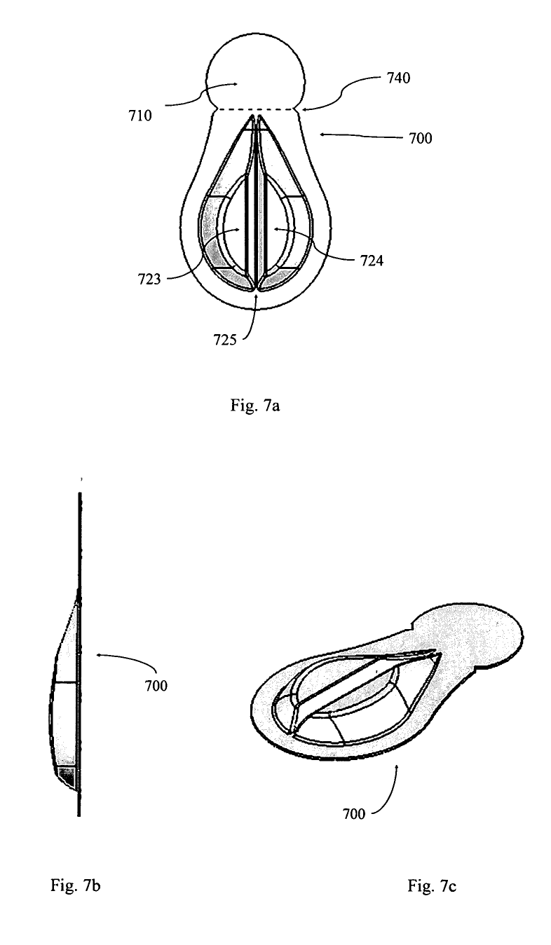

FIG. 7a illustrates sachet embodiment 700 with two compartments 723 and 724, the seal 725 dividing 723 from 724 being at the vertical midline. The sachet has a tear-off tab 710, removed at line of weakness 740. Topical compositions contained in compartments 723 and 724 can be mixed upon exit from the sachet. FIGS. 7b and 7c further illustrate sachet 700.

While amounts sufficient for multiple uses of the composition may be contained in the sachet disclosed herein, in one embodiment, the sachet is designed for a single use, i.e. a unit dose. As the unit dose can be applied to the entire keratinous surface of the user, for example, the entire skin, hair or nail surface, or can be a spot application, a unit dose can very from 0.1 gram to 30 grams. The perimeter or seals of the sachet can be made to create a dispensing outlet to allow the topical composition to be more precisely controlled when dispensing. This allows more precise dispensing but can also be used to require a reasonable dispensing force before product can be dispensed. By narrowing the outlet, the product can be kept from leaking out of the reservoir unless substantial force is applied to the reservoir when desired by the user to dispense the product.

While some variations may utilize a sachet that is opened prior to heating, the sachet generally will remain sealed during heating, since topical compositions or the active ingredients in 1 5 those compositions may degrade when subjected to air, oxygen, light, and/or microbes.

Advantages of the present invention utilizing a sachet include minimizing exposure of the composition to these degrading conditions both prior to heating and by the nature of the sachets having limited volume after heating and application to the keratinous tissue. This promotes efficacy of the composition and, or its active ingredients that may have been avoided in the past 20 due to their susceptibility to degradation. An additional benefit to heating of a sealed sachet, is that spillage into the device is eliminated. Not only is mess reduced, but since the device typically has some electronic aspect, introduction of liquid product into the device causing, malfunction of the components is minimized.

Preventing contact of the product with atmospheric gases also enhances stability of the topical composition. Thus, the sachet may be produced with limited or no headspace, and/or with a non-oxidizing gas other than air, such as nitrogen,

The sachet in some embodiments may be flexible, while in others it is rigid. Flexible sachets when placed in an appropriately shaped receptacle deform around a heat sink in the receptacle to allow for maximum efficiency of heat transfer to the sachet and ultimately to its contents. Flexible sachets can also be easily dispensed by squeezing the contents into the hand. A rigid blisterpack with a flexible film back cover can provide a disk that allows product to be dipped out with the fingers after the back cover sheet is removed.

Heat transfer to and from the sachet and its contents is important both during heating and during use because it determines how quickly the sachet and its contents warm and cool. Since heat transfer to the topical composition within a sachet is a function of the thermal resistance of the material comprising the sachet, choosing the sachet surface materials carefully allows the composition in the sachet to be heated in a desired time frame and can be maintained at a desired temperature in the receptacle for immediate use on keratinous tissue. For example, increasing the thickness of the sachet surface increases the thermal resistance of that surface. The higher thermal resistance reduces the rate of heat transfer through the surface to the composition. A sachet with thicker surfaces, then, will take longer to warm the composition to a specific temperature, but will retain the warmth of the composition for a longer period of time. Thermal resistance is also affected by a material's thermal conductivity. Materials with higher thermal conductivities can be used to decrease the surfaces' thermal resistance. Materials with lower thermal conductivities can be used to make surfaces with higher thermal resistance. The heat transfer area also affects thermal resistance of the sachet surface. As the area increases, the thermal resistance decreases. Larger heat transfer areas decrease the heating and cooling time of the sachets.

Combinations of materials comprising the same sachet are also contemplated. For example one surface of the sachet may have higher thermal resistance. This would reduce the heat lost to the user's fingers while the user holds the sachet. This surface would not feel as warm to the touch. Choosing a material with higher thermal conductivity, like a foil instead of a plastic, reduces the thermal resistance. A foil sachet will heat more quickly, but also cool quickly. Another means of reducing thermal resistance is by increasing the heat transfer area. Therefore, combinations of materials would provide thermally insulated surfaces, while less thermally resistant surfaces would allow for faster warming.

The sachet may comprise at least one surface that is coated or embedded with a volatile material. When the sachet is heated, the volatile material, such as perfume, scents the surrounding air.

Materials which change color or level of opacity when the topical composition has been warmed may optionally be used in sachet surfaces. This would result in a visual signal based in the sachet surfaces.

One embodiment of the sachet comprises a solid including powder, beads minerals, etc. In a further embodiment, one of the sachet surfaces may comprise an applicator for the topical composition. For example, the surface to be used as an applicator can comprise a portion that is a sponge-like material. This sponge-like portion will ensure that the topical composition is evenly applied to the user's keratinous tissue. Alternatively, the applicator can take the form of a brush, a dropper, or a syringe.

In another embodiment the sachet comprises topical compositions in combination with a substrate to effect cleaning, treating, or other uses. In a preferred embodiment, the composition is pre-combined with the substrate or towelette to form a wipe product, e.g., disposable wipe products, to be used for such purposes at a later time. In a preferred embodiment, the wipe product includes a nonwoven substrate impregnated with a topical composition inside the sachet.

The sachet may be made in a variety of shapes, including but not limited to pouches, packets, cells, blisters, thermoformed cups, capsules, ampoules, box-shapes, cones, balloon shapes, bellows shapes, flexible or rigid pouches with a multiplicity of sides, syringes, droppers, or vials. The sachet may be flat, rounded or combinations thereof.

Receptacle

The receptacle, comprising a heating system, comprises a cavity for releasably receiving the sachet. The heating system utilized herein comprises a heating element associated with a cavity wall. Alternatively, the heating element can be associated with a plurality of cavity walls. For example, in embodiments of the invention wherein multiple sachets are heated simultaneously, the heating element can extend from the cavity wall adjacent to the source of power, extending laterally along the walls of the cavity or completely about the entire internal surface of the receptacle. In an alternative embodiment the heating element and/or heat sink can be positioned in the center of the cavity such that a sachet can be placed on each side and heated simultaneously.

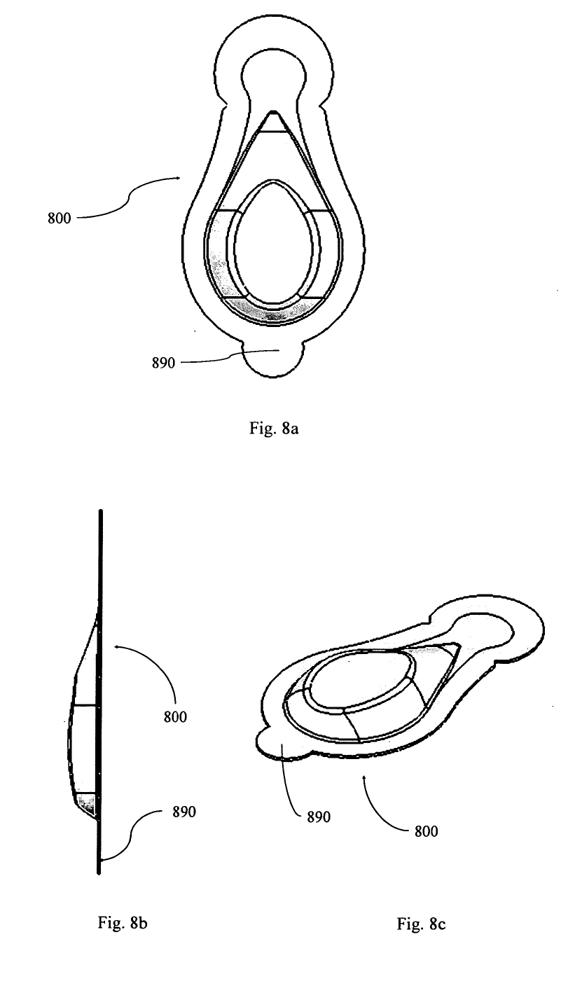

In one embodiment, the heating system is activated by means of a manual switch. In another embodiment, the heating system activates upon insertion of a sachet into the cavity of the receptacle thereby engaging a switch in communication with an internal wall of the cavity. The area of the sachet that engages the switch is referred to as a “contact point”. FIG. 8a shows sachet embodiment 800 with contact point 890, designed to activate the heating system by means of a switch located in the bottom of the cavity in some embodiments of the receptacle. FIG. 8b is a side view and FIG. 8c is a perspective view of sachet 800, both further illustrating contact point 890.

Other optional components contemplated for use in the heating system disclosed herein are, a heat sink in communication with the heating element, and temperature sensors, optionally in communication with the heat sink. Optionally, a thermal cutoff or fuse between the heating element and the power source can be used. Indicators comprising visual feedback mechanisms, auditory feedback mechanisms, tactile feedback mechanisms and combinations thereof, can be operatively associated with a temperature sensor, a monitor, and combinations thereof. A monitor can be used to control all functions of the heating systems including the indicator. The monitor can comprise a microprocessor, microcontroller, timer, or sensor as well as other elements.

In a further embodiment, the heating system activates upon insertion of a sachet having one or more protrusions on its exterior surface. The protrusions provide communication with switches located in the walls of the cavity. The protrusions can provide a “lock and key function” to avoid accidentally turning on the heating system by the insertion of foreign objects into the device, avoiding melting such objects and destroying the receptacle. In yet another embodiment, a sensor in the receptacle wall recognizes the presence of the sachet and activates the heating element. When the sachet is removed the heating element is deactivated and the receptacle ceases to provide warmth. Sensors can be based on changes in the resistance, capacitance, inductance, light transmission, force, pressure, voltage, current or other means when the sachet is present as opposed to when it is not. For example, a photodiode receiving light from a light emitting diode (LED) could be covered by the sachet, changing the output signal of the photodiode. This change in light intensity could be used to control the heater. A sachet having the right properties to change the desired sensor signal could provide a lock and key function.

Warming of the topical composition to its target temperature occurs in about 30 seconds to about 5 minutes, preferably in about 30 seconds to about 2 minutes. The target temperature of the composition is between about 30° C. and about 65° C., preferably between about 35° C. and about 65° C., more preferably between about 37° C. and about 60° C., most preferably between about 40° C. and about 55° C.

In an alternative embodiment, the heating system maintains the target temperature of the composition for a predetermined length of time. This length of time is from about 1 minute to about 1 hour minutes, preferably about 3 minutes to about 30 minutes.

A heat sink in contact with the heating element may optionally be used to transfer and distribute heat generated by the heating element. A temperature sensor may be in communication with the heat sink, and a monitor may be in communication with the temperature sensor. When the temperature sensor generates a signal that the heat sink is over its operational temperature for a designated period of time, the monitor can adjust or interrupt the power to the heat sink allowing it to cool, thereby lowering the temperature of the composition within the sachet into its desired temperature range. This interruption of power delivery to the heating element can function as a thermostat, a safety feature and a power saving feature. If the sachet has been heated for a predetermined length of time at a specific temperature without removal of the sachet, warming is discontinued until the sachet is removed and replaced or until the heating element is activated in some other way. If, on the other hand, the temperature sensor generates a signal that the heat sink is below its operational temperature, the monitor can adjust the power to the heat sink to raise heat, thereby raising the temperature of the composition within the sachet into its desired range.

Heating elements preferred for use in the invention disclosed herein are electrical resistance heaters. Examples include, resistive alloy wire or foil, thermally conductive plastics, carbon/silver inks printed onto polyester or other flexible materials, etched foil heaters, Positive Temperature Coefficient Heaters (PTC) (ceramic stones), thick film heaters, or electrical components that give off heat due to electrical resistance to current flow.

Specific types of resistive alloy wire or foil type electrical resistance heaters which can be selected are: rope/wire elements in metal tubes, rope/wire elements insert molded into heater assemblies, wire wound heaters, LCD heaters and ceramic fiber insulated wire heaters.

Other heater types suitable for use in the invention disclosed herein are: Peltier junction heaters, microwave emitters, radiant heating elements, light bulbs, and heat lamps.

Chemicals that, when combined result in an exothermic reaction, can also be utilized to produce the heat necessary to warm the composition. Examples include: heat of hydration reactions, anhydrous reactions, heat of solution and neutralization reactions, oxidation reactions, crystallization reactions, electrochemical heating, phase change material, condensation, etc.

Power itself can be supplied by various sources: AC or DC power mains, battery, solar power, combustible gases, chemical reaction, or any combination of these. Combustible gases useful in the present invention include those that are known for use in portable butane-fired hair curling devices.

In a further variation, the receptacle comprises an indicator, the indicator being operatively associated with the temperature sensor, monitor or timer. The indicator produces a signal when the topical composition reaches its target temperature or at the end of a predetermined period. The indicator can signal “warming”, “warm”, “ready”, “maintaining” “on” or “off”. The signal given by the indicator can be a visual signal, an audio signal or a combination or these.

Material selection and thermal resistance of the receptacle also affect its performance. For example, the heat sink, if present, would have low thermal resistance and should store minimal heat if it is to warm the sachet quickly. The walls of the receptacle cavity should be made of a material that is rated for the required temperatures and voltages that the receptacle could experience. Underwriters' Laboratories Inc. provides rating of these materials. In addition to necessary UL material ratings, the walls of the receptacle cavity can have higher thermal resistance to prevent heat loss to the ambient environment.

While heat sinks are commonly used in the receptacles disclosed herein, some examples do not require one. For example, a flexible film heater made of carbon and silver printed on polyester can quickly and efficiently heat a sachet if it is placed directly in communication with the sachet. Such heaters, produced by Conductive Technologies, Inc., York, Pa., are used in medical applications to heat intravenous fluids to body temperature. These heating systems are positive temperature coefficient heaters as well.

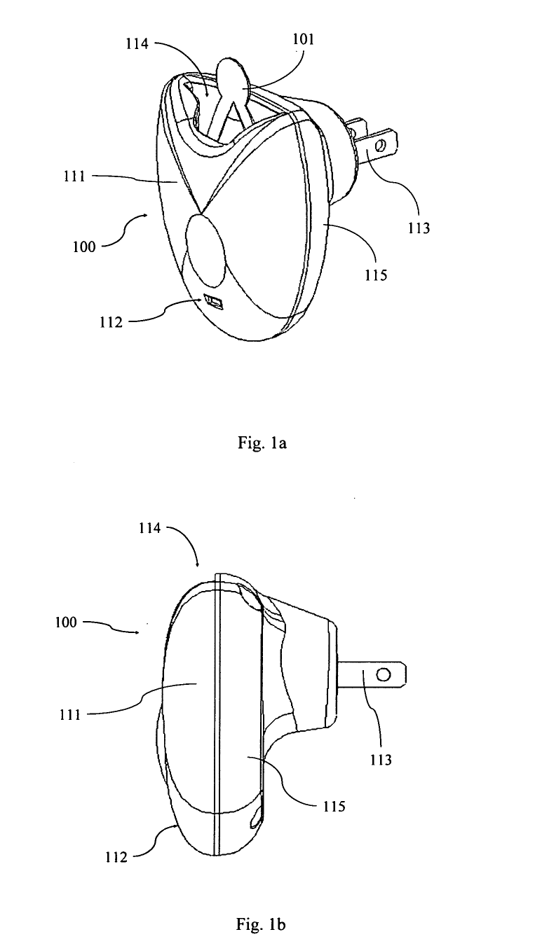

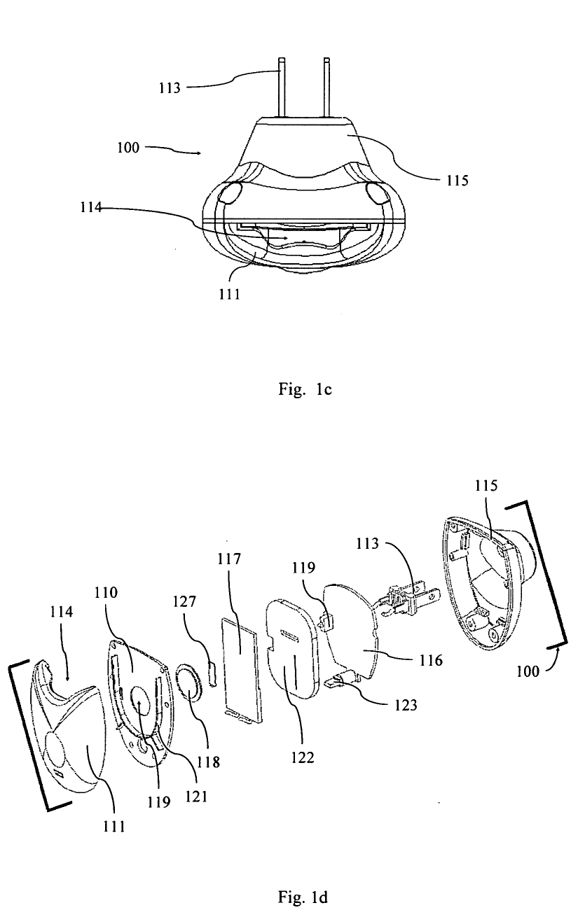

FIG. 1a illustrates an embodiment of the receptacle 100 disclosed herein. The receptacle 100 shows a sachet 101 in place inside the cavity 114 of the receptacle. In this example, the power is U.S. household electric current (110-120V) conducted through the plug 113.

FIG. 1b illustrates a right side view of the embodiment of FIG. 1a with no sachet in place. FIG. 1c is a top plan view of the receptacle of FIG. 1a, showing front housing 111, cavity 114, plug 113 and rear housing 115.

FIG. 1d is an exploded view of FIG. 1a, without the sachet in place. The front housing 111 and the rear housing 115 enclose a printed circuit board 116 that contains a microcontroller. The microcontroller stores and executes software instructions that govern all other components. A heating element 117 is operatively associated with a heat sink 118. In this embodiment, a switch 119, is depressed upon insertion of the sachet to activate the heating element 117. Inner plate 110 forms the back wall of the cavity 114 for containing the sachet. A sachet rest 121 is mounted on the front surface of inner plate 110. Light emitting diode (LED) 123 signals whether receptacle 100 is in ramping mode or holding mode, discussed below. Item 122 is silicone rubber insulation, protecting the printed circuit board (PCB) from the heating element. Resistance temperature detectors (RTD) 127, which measure the temperature of the heating element or heat sink. A thermal cutoff (TCO) not shown, interrupts the flow of current to the heating element in the event that the temperature measured by the TCO exceeds its rated operating temperature. In this embodiment, the TCO used in the device has a UL rated function temperature of 120° C. Item 119, mentioned above, is a switch. The switch extends into the cavity 114 that receives the sachet. Upon insertion of the sachet, the switch 119 is triggered, activating the heating element 117. Plug, 113 connects the heating element 117 to electric current.

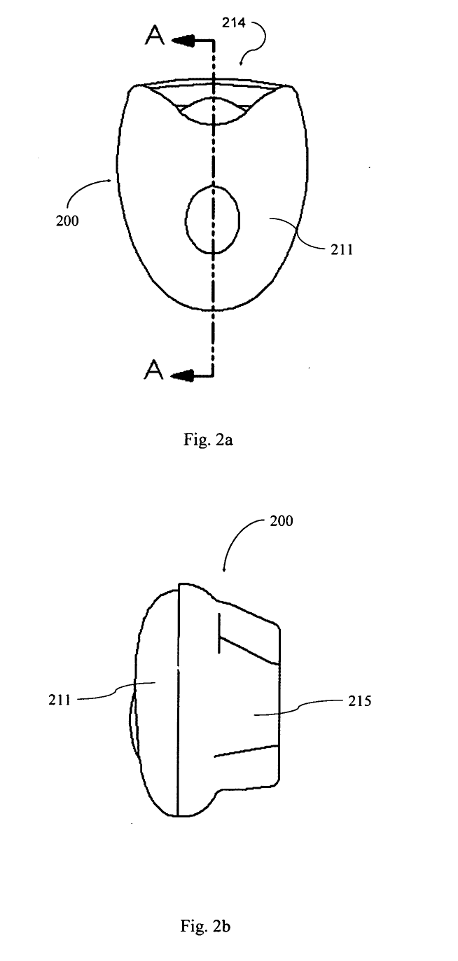

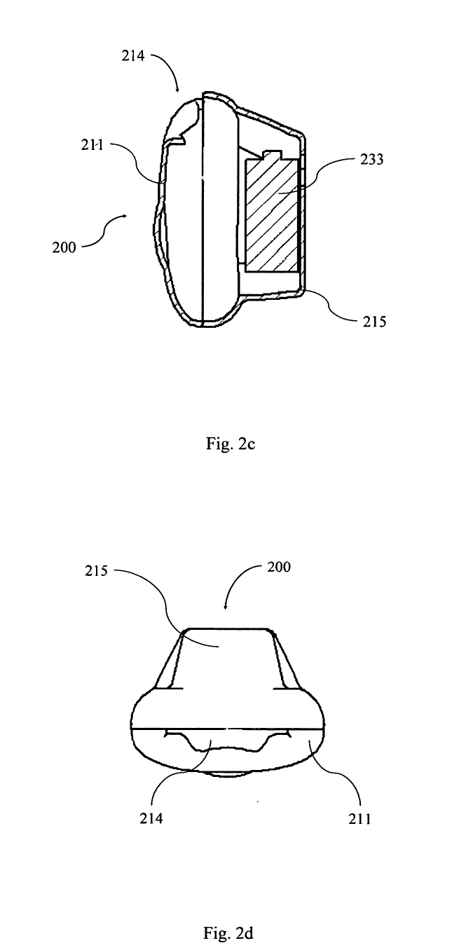

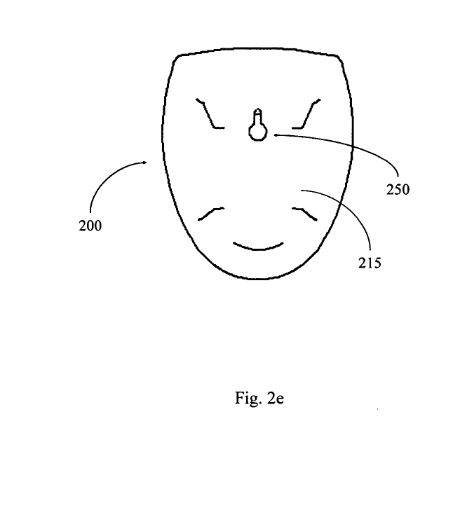

FIG. 2a illustrates a second embodiment of the receptacle 200 disclosed herein. In this example, the receptacle contains a battery 233 shown in FIG. 2c. The front housing 211 of the receptacle 200 encloses the front portion of cavity 214.

FIG. 2b illustrates a right side view of the embodiment of FIG. 2a showing rear housing 215 of the receptacle 200.

FIG. 2c is a cross sectional view of FIG. 2a taken along line A-A. The front housing 211 and the rear housing 215 enclose cavity 214 and the heating system (not shown).

FIG. 2d is a top plan view of the receptacle 200 of FIG. 2a.

FIG. 2e shows the rear housing 215 of receptacle 200. Item 250 is an opening in the rear housing which allows mounting of the receptacle via a suitable hanger.

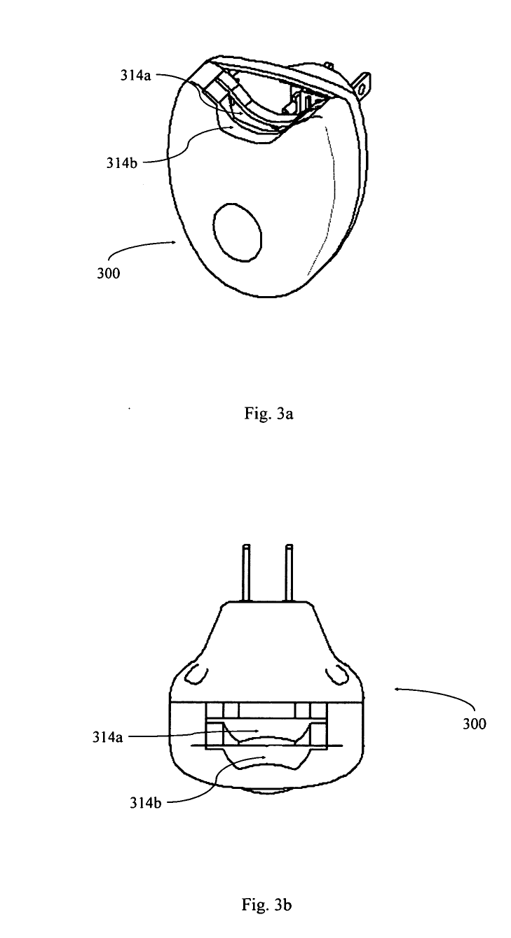

FIG. 3a shows a third embodiment of the receptacle disclosed herein. This receptacle 300 is designed to heat two sachets simultaneously. These sachets can comprise topical compositions which are the same or different. Items 314a and 314b are two areas within the receptacle cavity for sachet insertion.

FIG. 3b is a top plan view further illustrating receptacle 300.

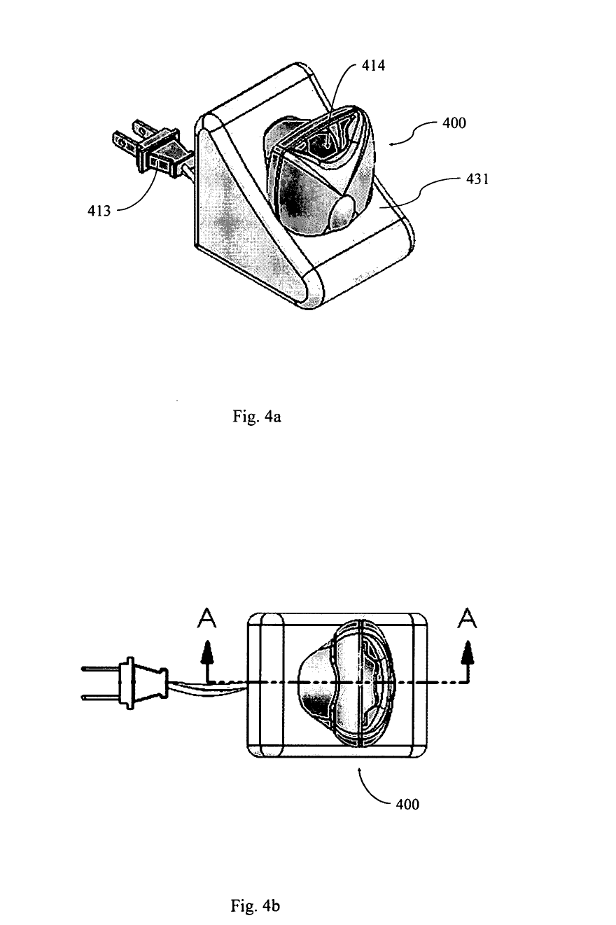

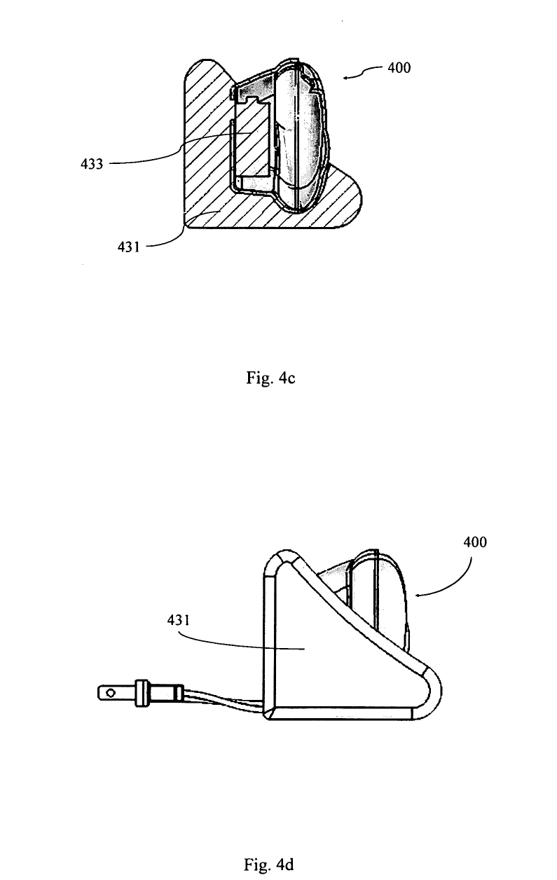

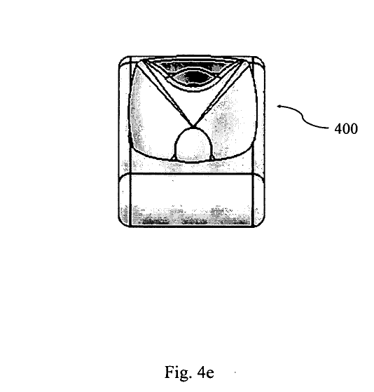

FIG. 4a shows another embodiment in which a receptacle 400 is mounted for recharging on a docking station 431 that is connected to electrical current via plug 413. FIG. 4c is a cross sectional view of the receptacle with docking station taken along line A-A of FIG. 4b which is a top plan view of 400. This cross section shows a battery 433 which can be recharged while mounted on the docking station 431. This embodiment allows the use of rechargeable batteries, so that, when its batteries are fully charged, the receptacle can be used in remote locations. FIG. 4d shows a left side view, further illustrating receptacle 400.

The heating cycle may optionally, consist of two distinct modes, a ramping mode, when the sachet and its contents are being warmed and a holding mode. During the holding mode the sachet and its contents are held at a constant temperature. If the sachet is removed at any time during the heating cycle, the heater will be turned off.

Referring to FIG. 1 as an illustration, during the ramping mode, a Resistance Temperature Detector (RTD) 127 shown in FIG. 1d measures the temperature at the heater to heat sink junction. The microprocessor, within the Printed Circuit Board 116, calculates a ramp rate for the heater, such that the sachet and its contents attain a temperature of 50° C. in 3 minutes.

After the ramping mode is complete, the holding mode is entered. During the holding mode, the temperature of the heater is set to 60° C. for 15 minutes. If the sachet is not removed after 15 minutes, the heater turns off. The user must remove the sachet and re-insert if the receptacle is to start heating again. Should the sachet be re-inserted, the heating cycle will repeat.

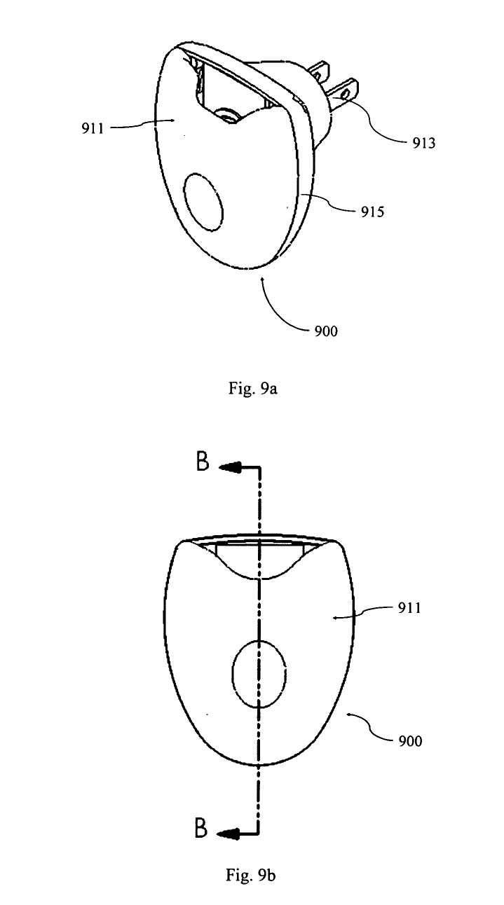

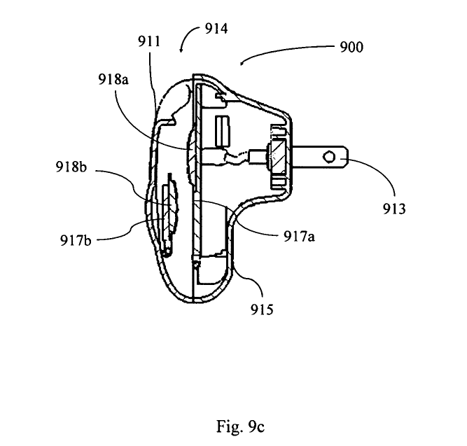

FIG. 9a is a perspective view of a fifth embodiment of the receptacle 900 having two heating elements and two heat sinks, front housing 911, rear housing 915, and plug 913. FIG. 9b is a front plan view of receptacle 900 showing front housing 911. FIG. 9c is a cross sectional view of receptacle 900 taken along line B-B of FIG. 9b. Items 917a and 917b are heating elements. Items 991a and 918b are heat sinks for the heating elements to which they are adjacent.

Materials which change color or level of opacity when the topical composition has been warmed may optionally be used in the receptacle. This would result in a visual signal based in the receptacle.

Method

The method of treating keratinous tissue comprises as a first step, warming a topical composition contained in a sachet using a receptacle, as described above. The power source supplies sufficient energy to the heating system to warm the composition inside the sachet to a target temperature sufficient to provide easy application to the keratinous tissue but not so fluid as to run off the surface when applied. Generally the temperature is from about 30° C. to 65° C. The warmed composition is removed from the sachet and applied to keratinous tissue.

The topical composition can be chronically applied. By “chronic topical application” is meant continued topical application of the composition over an extended period, preferably for a period of at least about one week, more preferably for a period of at least about one month, even more preferably for at least about three months, even more preferably for at least about six months, and more preferably still for at least about one year. Typically, applications would be on the order of about once per day; however application rates can vary from about once per week up to about three or more times per day.

Regimen

A skin care regimen, using a receptacle as described above, comprises selecting a sachet from a sachet assortment, inserting the sachet into the receptacle for releasably receiving the sachet, and activating the heating system operatively associated with the receptacle The topical composition is then warmed in the sachet. After warming, the sachet is opened and the warmed composition is applied to a user's skin. A schedule of chronic application of the topical composition, similar to that outlined above, can be used in the skin care regimen.

All documents cited in the Detailed Description of the Invention are, in relevant part, incorporated herein by reference; the citation of any document is not to be construed as an admission that it is prior art with respect to the present invention.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

1. A method for treating keratinous tissue, said method comprising:
a) warming a topical composition contained in a sachet using a receptacle, said receptacle comprising:
i) walls defining a cavity for releasably receiving at least one said sachet;
ii a heating system associated with one or more of said walls of said cavity;
iii a power source operatively associated with said heating system for supplying sufficient energy to said heating system to warm said composition to a target temperature;
b) applying said warmed topical composition to said keratinous tissue.

2. The method according to claim 1 wherein said composition comprises at least one active ingredient selected from the group consisting of: desquamation actives; anti-acne actives; anti-wrinkle actives; anti-atrophy actives; anti-oxidants; radical scavengers; chelators; flavonoids; anti-inflammatory agents; anti-cellulite agents; tanning actives; skin lightening agents; antimicrobial and antifungal actives; sunscreen actives; conditioning agents; water-soluble vitamins; particulate materials; sugar amines; vitamin B3 compounds; retinoids; peptides; phytosterols; hexamidines and their derivatives; dialkanoyl hydroxyproline compounds; salicylic acid compounds; n-acyl amino acid compounds; dehydroacetic acid, its isomers, salts, and derivatives thereof; skin firming agents and mixtures thereof.

3. The method of claim 1 wherein said topical composition has a viscosity between 10,000 and 500,000 cps at its target temperature.

4. The method of claim 4 where upon warming the composition, its viscosity stays within 20% of its viscosity prior to warming.

5. The method of claim 4 wherein said composition is solid at ambient temperature.

6. The method according to claim 1 wherein the topical composition comprises a water in oil emulsion.

7. The method of claim 1 wherein the topical composition comprises at least 1% waxy materials.

8. A receptacle for warming a topical composition contained in a sachet, said receptacle comprising:
i) walls defining a cavity for releasably receiving at least one said sachet;
ii) a heating system associated with one or more of said walls of said cavity, wherein said heating system has the capability of achieving and maintaining a target temperature of the topical composition; and
iii) a power source operatively associated with said heating system for supplying energy to said heating system.

9. The receptacle according to claim 8 wherein said heating system is controlled by a monitor.

10. The receptacle according to claim 9 wherein said receptacle further comprises an indicator, said indicator being operatively associated with said monitor, to produce a signal at said target temperature.

11. The receptacle according to claim 8 said receptacle further comprising a temperature sensor operatively associated with an indicator, said indicator producing a signal at said target temperature wherein said signal is selected from the group consisting of visual, audio and tactile signals and combinations thereof.

12. The receptacle according to claim 8 wherein said heating system is activated by insertion of said sachet into said cavity.

13. The receptacle according to claim 8 wherein said heating system is activated by a manual switch.

14. The receptacle of claim 8 wherein said heating system warms said topical composition to its target temperature in about 30 seconds to about 5 minutes.

15. The receptacle of claim 8 wherein said heating system maintains said target temperature of said composition for predetermined period.

16. The receptacle according to claim 8 wherein said heating system comprises:
a) a heating element associated with said cavity wall adjacent to said power source;
b) a heat sink operatively associated with said heating element;
c) a temperature sensor in communication with said heat sink; and
d) a monitor in communication with said temperature sensor, said monitor capable of interrupting the delivery of energy from said power source to said heating element at a predetermined period.

17. The receptacle of claim 8 wherein said power source is supplied by AC/DC house current, battery, solar power, combustible gases, chemical reaction, and any combinations thereof.

18. The receptacle of claim 8 further comprising a timer.

19. The receptacle of claim 8 said receptacle comprising an element selected from the group consisting of sensors, switches and combinations thereof located in said walls of said cavity, said elements being capable of communication with one or more protrusions or contacts points on a surface of said sachet.

20. A kit for treatment of keratinous tissue, said kit comprising:
a) at least one topical composition said composition comprising at least one active ingredient;
a) at least one sachet comprising surfaces defining at least one chamber for containing said composition; and
b) a receptacle for releasably receiving said at least one sachet, said receptacle comprising:
i) walls, said walls defining a cavity for releasably receiving said sachet;
ii) a heating system associated with one or more of said walls; wherein said heating system has the capability of achieving and maintaining a target temperature of the topical composition; and
iii) a power source operatively associated with said heating system.

21. The kit of claim 20 wherein said sachet contains a unit dose of said topical composition.

22. The kit of claim 20 wherein said sachet contains from about 0.5 ml to about 5 ml of said topical composition.

23. The kit of claim 20 wherein said sachet contains a unit dose of said topical composition.

24. The kit of claim 20 wherein said sachet contains from about 0.5 ml to about 5 ml of said topical composition.

25. The kit of claim 20 wherein said heating system comprises a heating element associated with said cavity wall and a heat sink operatively associated with said heating element, and wherein said sachet when inserted into said receptacle deforms around said heat sink.

26. A skin care regimen comprising:
a) selecting a sachet from a sachet assortment, said sachet comprising at least one chamber containing a skin care composition;
b) inserting said sachet into a receptacle for releasably receiving said sachet, said receptacle comprising:
i) walls, said walls defining a cavity for releasably receiving said sachet;
ii) a heating system associated with one or more of said walls; and
iii) a power source operatively associated with said heating system;
c) activating said heating system, thereby warming the composition contained in said sachet;
d) opening said sachet after warming;
e) applying said warmed composition to a user's keratinous tissue at least one time per day.
